(19) United States
(12) Patent Application Publication
(10) Pub. No.: US 2010/0317000 A1
(43) Pub. Date: Dec. 16, 2010

(54) METHOD FOR DIAGNOSING BLADDER CANCER BY ANALYZING DNA METHYLATION PROFILES IN URINE SEDIMENTS AND ITS KIT

(75) Inventor: Jingde Zhu, Shanghai (CN)

Correspondence Address: LERNER, DAVID, LITTENBERG,;KRUMHOLZ & MENTLIK, 600 SOUTH AVENUE WEST, WESTFIELD, NJ, 07090, US

(73) Assignee: SHANGHAI CANCER INSTITUTE, Shanghai (CN)

(21) Appl. No.: 12/670,491

(22) PCT Filed: Jul. 23, 2008
(86) PCT No.: PCT/CN2008/071725

§ 371 Date: Aug. 3, 2010

(30) Foreign Application Priority Data
Jul. 23, 2007 (CN) 200710044106.1

(51) Int. Cl. C12Q 1/68

(52) U.S. Cl. 435/6

(57) ABSTRACT

The present invention provides a method for detecting bladder cancer in a subject, comprising the following steps: (a) providing urine sediment sample from said subject; (b) determining methylation pattern of a given sequence within the promoter CpG islands of one or more genes (known as “gene” infra) in the samples; (c) comparing the methylation pattern from said subject with that from normal subject, wherein the hypermethylation of one or more of genes indicates that said subject is suffering from bladder cancer. The present invention also provides a kit for diagnosing bladder cancer.

### FIELD OF THE INVENTION

The present invention relates to kits and methods for diagnosing bladder cancer by detecting the altered DNA methylation pattern of the specific sequences in the promoter CpG island of genes in urine sediments from individuals with bladder cancer (including pre-neoplastic stages) as compared to that from the normal individuals (or individuals without bladder cancer).

### BACKGROUND OF THE INVENTION

Having the genetic blueprint for human and increasing number of model organisms available has ushered in a new era for the genetic makeup and functional elucidation in development and disease states, which chiefly concerns analysis and annotation of the epigenetic information that inheritable through cell division without changes in DNA sequence. The epigenetics consists of DNA methylation (cytosine [CpG] methylation), non-coding RNA, histone modification, and chromatin remodeling. This interface sits between the genetic blueprints stored in genomic DNA sequences and phenotypes dictated by the pattern of gene expression. It more readily responds to the changing environment than its sequence based genetic counterparts [1]. Addition of the methyl group at cytosine ring within 5′-CpG-3′ sequence (FIG. 1) was carried out by one of the three DNA methyl transferase genes (DNMT1, DNMT3a, and DNMT3b) using S-adenosyl methionine as the methyl donor. The DNA methylation pattern in the parental cells can be faithfully duplicated and distributed into daughter cells in a fashion similar to the semi-conservative replication mechanism for the genetic information. DNA methylation is the key mechanism determining the transcriptional memory. The pattern of DNA methylation changes markedly during the early embryonic development as well as germ cell maturation (the epigenetic reprogramming), and moderately throughout the life of living organisms. Abnormal epigenetic homeostatic mechanism would lead to accumulation of the epigenetic lesions, and ultimately the various diseases states, including cancer[2].

Cancers are extremely complex diseases with extensive genetic and epigenetic defects. The defects vary with both types of cancer and individual patients[3]. DNA methylation based on the enzymatic process to add the methyl group at the fifth carbon of cytosines within the palindromic dinucleotide 5′-CpG-3′ sequence (DNA methylation)(FIG. 1) is the best studied epigenetic mechanism and the focus of cancer epigenetic study.

Over 85% CpG dinucleotides are spread out in the repetitive sequences with the transcription-dependent transposition potential. They are heavily hypermethylated/transcription-silenced, a state required for the genome integrity. The extensive hypomethylated state of genome in cancer cells leads to the transcription of the repetitive sequences and enhancement of transposition activity [2,4], which, subsequently, increases genomic instability and transcription of proto-oncogenes [5,6]. The remaining CpG are clustered within the short DNA regions (approximately, 0.2 to 1 kb in length), known as “CpG island”. Approximately 40-50% of the genes have CpG island within or around the promoter, indicating that transcription of these genes can be regulated by DNA methylation-mediated mechanism. Although mostly unmethylated in normal cells, some of them are often hypermethylated and the transcriptional silencing, including the tumor suppressor genes, DNA repairing genes, cell cycle control genes, anti-apoptotic genes, and the like.

The critical role of the epigenetic abnormality at the early stage of carcinogenesis can be presented as loss of genetic imprinting (LOI). For example, overexpression of the genetic imprinting gene IGF2 can promote cell proliferation, and LOI of which was found in normal-appearing colonic epithelium of patients with colorectal cancer, and LOI of this gene in circulating leucocytes is a crucial feature of subjects susceptible to colon cancer[7]. The hypermethylation/transcription silencing of the tumor suppressor and DNA repairing genes was common at the pre-neoplastic stage[8,9]. For instance, the hypermethylated p16ink4A (tumor suppressor gene) and MGMT (DNA repairing gene) were found in the sputum DNA[8]. Abnormal epigenetic state can also result in abnormal proliferation of stem cells, promoting carcinogenesis. The association of H. pyrio infection with the aberrant DNA methylation of a given set of genes suggests detection of DNA methylation provide a pre-warning [10]. Therefore, the tumor warning value of analysis of the DNA methylation of the peripheral DNA (serum, stool, sputum, and urine sediments as the sample sources) from the population at high risk for cancer has been also seriously considered.

In terms of incidence, Bladder cancer is the fourth most common cancer in men and the eighth most common cancer in women in the United States[11]. Its incidence increases dramatically in industrializing China[12]. Although over 70% patients suffering from the superficial lesions could be cured surgically, still 50-70% of those patients will return with more severe conditions and poor prognosis. The bladder cancers at similar pathologic grades and stages have variable clinical behaviors[15], illustrating the substantial deficiency of the exsting system. The gold standard for bladder cancer diagnosis is cystoscopy along with biopsy, but the misdiagnosis rate can be up to 10-40% [16-18]. Urine cytology is a non-invasive detection method with high specificity, but suffered from the low sensitivity for Ta, G1, and T1 bladder cancers [19]. The attempt of use of genetic detection of cellular DNA in urine sediments in diagnosing bladder cancer has involved TP53 gene mutations, loss of heterozygosity, microsatellite instability, and E-cadherin promoter polymorphism (51) [20,21]. A method of seeking for chromosomatic abnormality by in situ cell hybridization in urine sediments is reported to detect 68.6% bladder cancer with 77.7% specificity (http://www.urovysion.com). Many attempts using protein marker were reported [22,23]. Although the assay for protein MNP22 in urine seems more sensitive than the urine cytology, it suffered from a substantial deficiency of the high level of the said protein in patients with benign urinogenital diseases such as hematuria, urocystitis, renal calculi, or urinary tract infections[24]. Therefore, there is still a need for developing a more sensitive and specific method for diagnosing bladder cancer and other types of urinogenital cancers, especially at the early stage thereof.

DNA methylation analysis methods generally rely on methylation modification of the original genomic DNA before any amplification step, comprising using the methylation-sensitive restriction enzyme digestion and bisulphite treatment [25]. The latter one exploited the sharp difference in the sensitivity to the bisulphite-mediated deamination (C to U conversion) between cytosine and methylated cytosine residues, which enable detection of as few as 1-10 tumor cells among 10⁴ normal cells[25]. Attempts of assaying methylation patterns of genes in bodily fluids, including bronchoalveolar lavage fluid, stool, serum, or plasma and urine sediments, for in vitro detection of cancer have been intensively reported. Other methods of detecting DNA methylation pattern include methylation-specific enzyme digestion, methylation-sensitive single nucleotide primer extension (MS-SnuPE) [26], restriction landmark genomic scanning (RLGS) [27], differential methylation hybridization (DMH) [28], BeadArray platform technology (Illumina, USA)[29], and base-specific cleavage and mass spectrometry (Sequenom, USA)[30], as well as those under development or to be developed.

### SUMMARY OF INVENTION

To achieve the above purpose, the present inventor has carried out extensive research and firstly discloses the difference of DNA methylation patterns between subjects with bladder cancer and those without bladder cancer, and detection of which may be used to determine bladder cancer in a subject. The method comprises the following steps:

(a) providing urine sediment sample from said subject;

(b) determining methylation pattern of one or more genes in the urine sediments, wherein said genes are selected from a group consisting of ABCC13, ABCC6, ABCC8, ALX4, APC, BCAR3, BCL2, BMP3B, BNIP3, BRCA1, BRCA2, CBR1, CBR3, CCNA1, CDH1, CDH13, CDKN1C, CFTR, COX2, DAPK1, DRG1, DRM, EDNRB, FADD, GALC, GSTP1, HNF3B, HPP1, HTERT, ICAM1, ITGA4, LAMA3, LITAF, MAGEAI, MDR1, MGMT, MINT1, MINT2, MT1GMT, MINT1, MINT2, MT1A, MTSS1, MYOD1, OCLN, p14ARF, p16INK4a RASSF1A, RPRM, RUNX3, SALL3, SERPINB5, SLC29A1, STAT1, TMS1, TNFRSF10A, TNFRSF10C, TNFRSF10D, TNFRSF21, and WVVOX;

(c) comparing methylation pattern of said genes in the urine sediment sample from said subject with that from normal subject, wherein the hypermethylation of one or more of genes indicates that said subject is suffering from bladder cancer.

The present invention further provides the procedures and standards for methylation pattern analysis and determining bladder cancer in a subject. The methods and standards will be used in diagnosing, prognosing, and monitoring the recurrence, and determining whether the tumors have been surgically removed. Other advantages and features of the present invention have been further disclosed in the following specific embodiments with reference to the accompanied figures.

### BRIEF DESCRIPTION OF DRAWINGS

FIG. 1 provides a flow chart of cytosine (CpG) methylation. In FIG. 1A, DNA methyltransferases (DNMT) 1, 3a, or 3b catalyzes the addition of a methyl group (the circled CH₃) at position 5 of the pyrimidine ring of the cytosine nucleotide by using S-adenosyl methionine (SAM-CH₃) as a methyl donor. In FIG. 1B, a C-to-T transition is initiated by sulfonation of cytosine (1, cytosine to cytosine sulfonate), then hydrolytic deamination occurs (2, cytosine sulfonate to uracil sulfonate), with the process concluded by alkali desulfonation (3, uracil sulfonate to uracil). Methylated cytosine resists this chemical treatment; thus, methylated versus unmethylated CpG can be detected by a subsequent polymerase chain reaction (PCR), including methylation-specific PCR.

FIG. 2 shows the analysis results of methylation specific PCR of 20 genes and sequencing verification.

This figure shows the electrophoretogram of MSP data of the representative methylation state and its sequencing verification. The number above each lane is the Identification Number of patient, cell lines (5637, T24, and SCaBER). M Sss1 indicates the result of normal liver tissue DNA modified by methylation by M Sss1 methyl transferase in a tube used as positive control. Gene names are listed above each panel. The wild-type sequences and the sequences of representative PCR products cloned from T vectors are aligned.

FIG. 3 shows the MSP analysis results of 11 valuable genes in 15 tumor tissue samples and 9 urine sediment samples. FIG. 3A illustrates the electrophoretogram of the MSP results, the involved gene is indicated on the top right corner of each panel. As a loading reference, the electrophoretogram of non-methylated MSP product of CFTR gene (marked as CFTRu) is shown.

Note: Ur: urine sediment, T: tumor tissue, G XX: No. of clinical samples, BJ, bisulphate-treated DNA derived from a normal fibroblast cell line, used as control of non-methylated DNA template. H₂O: control without DNA template. M. Sss I: positive control of methylated template of methylated DNA derived from normal liver tissue in a tube.

FIG. 3B summarizes the results from analysis of 9 pairs of the matched tumor tissues and urine sediments. The filled boxes indicate the methylated targets, and the empty boxes indicate the unmethylated targets.

FIG. 3C shows a histogram of the matching profile of the DNA methylation patterns in the matched tumor tissues and urine sediments.

Y axis: the percentage of methylation targets in a subgroup. T/Ur: commonly methylated in both tumor tissues and urine sediments; T; only methylated in tissues, and Ur: only methylated in urine sediments. The number of events and (percentage) are shown at the top of each column.

FIG. 4 shows the gene methylation state in urine sediments from patients with bladder cancer and patients with non-cancerous urinogenital lesions. The lower panel describes the methylation frequency (y axis, %) of each gene (x axis) in the urine sediments from patients with bladder cancer (column 2) and patients with non-cancerous urinogenital lesions (column 3, FIG. 4A). CI (Confidence Index): The values of each gene within 95% confidence interval are presented as a perpendicular line on the panel. The positions of p values of <0.01 and <0.05 are indicated as their methylation states can be used as a marker for bladder cancer.

FIG. 5 shows the ROC (RECEIVER OPERATING CHARACTERISTICS) values of the sensitivities and specificities of the informative gene sets for bladder cancer detection. Both the sensitivity (%, Column 4, in FIG. 5A) and specificity (%, Column 5, FIG. 5A) of each gene set were calculated and plotted.

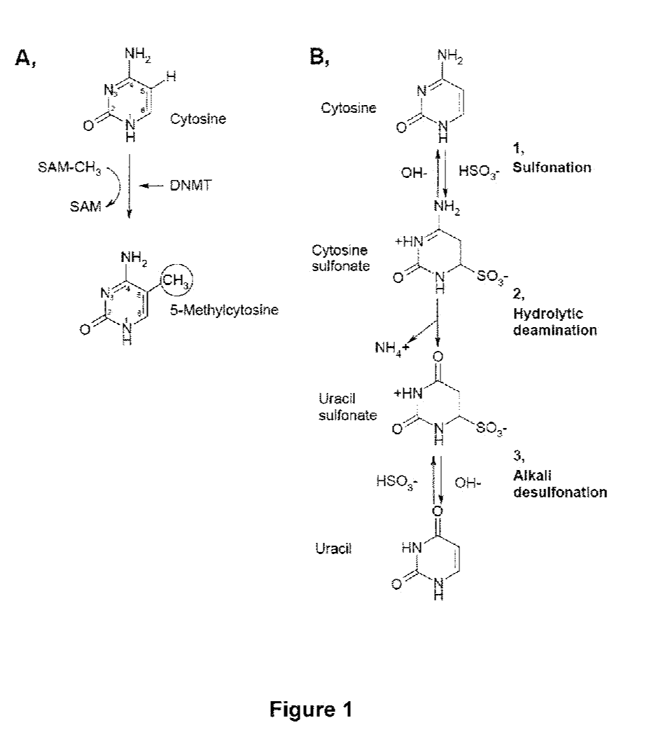

### DETAILED DESCRIPTION OF EMBODIMENTS

In one aspect, the present invention provides a method for detecting bladder cancer in a subject, comprising the following steps:

(a) providing a urine sediment sample from said subject;

(b) determining the methylation pattern of one or more genes in the urine sediments, wherein said genes are selected from a group consisting of ABCC13, ABCC6, ABCC8, ALX4, APC, BCAR3, BCL2, BMP3B, BNIP3, BRCA1, BRCA2, CBR1, CBR3, CCNA1, CDH1, CDH13, CDKNIC, CFTR, COX2, DAPK1, DRG1, DRM, EDNRB, FADD, GALC, GSTP1, HNF3B, HPP1, HTERT, ICAM1, ITGA4, LAMA3, LITAF, MAGEA1, MDR1, MGMT, MINT1, MINT2, MT1GMT, MINT1, MINT2, MT1A, MTSS1, MYOD1, OCLN, p14ARF, p16INK4a, PTCHD2, RASSF1A, RPRM, RUNX3, SALL3, SERPINB5, SLC29A1, STAT1, TIMP3, TMS1, TNFRSF10A, TNFRSF10C, TNFRSF10D, TNFRSF21, and WWOX;

(c) comparing the methylation pattern of one or more genes in the sample from said subject with that in the sample from normal subject, wherein the hypermethylated state in one or more genes indicates that said subject suffered from bladder cancer.

As used herein, the term “sample” in the context of the present invention is defined to include any sample obtained from any individual which is proper to test for DNA methylation, for example, those samples taken from the subjects with urinogenital symptoms. The term “urine sediment” has the meaning well known by a person skilled in the art, which includes the epithelial cells exfoliated from urethra, and etc. The cytological analysis of urine sediment has been used in clinical diagnosis of bladder cancer, since cells from bladder tumors are often exfoliated into urine sediment.

The sample being used in the present invention may also be the established bladder cancer cell lines, such as T24 (ATCC number: HTB-4), SCaBER (HTB-3), and 5637(HTB-9).

The present method is applicable to determine the urinogenital cancer. Said urinogenital cancer may include, for example, bladder cancer, prostate cancer, and kidney cancer. (Other types of cancer whose cells can be present in urine may also be detected by the present method. As a result, the “urinogenital cancers” are also included in the scope of the present invention.

The term “subject” as used herein includes, but not limited to, mammal, such as human.

The term “methylation” and “hypermethylation”, used interchangeably herein, are defined as the presence or high methylation of CpG loci within a gene sequence, most often within the promoter of a gene. When MSP is used, the tested DNA (gene) region can be considered to be hypermethylated if a positive PCR result is obtained from a PCR reaction using methylation-specific primers. Using Real-time Quantitative Methylation-Specific PCR, the hypermethylated state can be determined according to the statistically significant difference in comparison with the relative value of the methylation state of the control sample.

The basis of the present invention lies in that the methylation profiling of CpG sequence (for example, the region within the promoter CpG island of a tumor related gene, known as gene infra) from individuals suffering from bladder cancer is different from normal individuals or those whithout bladder cancer. As a result, the methylation state of one or more of the following genes may be used as an indicator of presence of bladder cancer in the subject. These genes may be selected from a group consisting of ABCC13, ABCC6, ABCC8, ALX4, APC, BCAR3, BCL2, BMP3B, BNIP3, BRCA1, BRCA2, CBR1, CBR3, CCNA1, CDH1, CDH13, CDKNIC, CFTR, COX2, DAPK1, DRG1, DRM, EDNRB, FADD, GALC, GSTP1, HNF3B, HPP1, HTERT, ICAM1, ITGA4, PTCHD2, LAMA3, LITAF, MAGEA1, MDR1, MGMT, MINT1, MINT2, MT1A, MTSS1, MYOD1, OCLN, p14ARF, p16INK4a, PTCHD2, RASSF1A, RPRM, RUNX3, SALL3, SERPINB5, SLC29A1, STAT1, TIMP3, TMS1, TNFRSF10A, TNFRSF10C, TNFRSF10D, TNFRSF21, and WWOX.

More particularly, the hypermethylation state of any gene selected from a group consisting of SALL3, CFTR, ABCC6, HPR1, RASSF1A, MT1A, RUNX3, ITGA4, BCL2, ALX4, MYOD1, DRM, CDH13, BMP3B, CCNA1, RPRM, MINT1, and BRCA1, in the urine sediment indicates that said subject is suffering from bladder cancer.

The methylation pattern of cellular DNA in the urine sediments may be determined by any techniques that are known (e.g. methylation-specific PCR(MSP) and Real-time Quantitative Methylation-Specific PCR, Metylite) or are under developing and to be developed. After bisulfite treatment, the unmethylated cytosines are converted to uracils, while the methylated cytosines remain unconverted. Subsequently, the DNA methylation state in the subject DNA is determined by amplifying the DNA after bisulfite treatment using primers capable of distinguishing methylated DNA from unmethylated DNA (30). This PCR approach, known as MSP can be used to detect small amount of tumor cells from a clinical sample with many normal cells with the proviso that the methylation state of the indicated DNA region (gene) in normal cells is opposite to that in tumor cells. It is possible to identify 1 tumor cells from 10,000 normal cells by using MSP.

It is preferred to use quantitative methylation-specific PCR (QMSP) in detection of methylation level. This method is based on the continuous optical monitoring of a fluorogenic PCR, which is more sensitive than the MSP method (31). It is a high-throughput technique and avoids analyzing its result by electrophoresis. The methods for designing primers and probes are known to the skilled in the art.

Additional useful techniques include methylation-specific enzyme digestion, bisulfite DNA sequencing, methylation-sensitive single nucleotide primer extension (MS-SnuPE) [26], restriction landmark genomic scanning (RLGS) [27], differential methylation hybridization (DMH) [28], BeadArray platform technology (Illumina, USA) [29], and a base-specific cleavage/mass spectrometry (Sequenom, USA)[30], and etc.

For a large sample analysis (comprising being compared with normal and/or non-cancerous subject), the methylation patterns of multiple tumor related genes are obtained, that is, it is possible to detect bladder cancer or other urinogenital cancer (prostate cancer or kidney cancer) in a subject by measuring methylation state of the gene sets.

The present invention also provides a kit for bladder cancer detection, comprising:

(a) means for measuring methylation pattern of one or more genes in the urine sediments, wherein said genes are selected from a group consisting of ABCC13, ABCC6, ABCC8, ALX4, APC, BCAR3, BCL2, BMP3B, BNIP3, BRCA1, BRCA2, CBR1, CBR3, CCNA1, CDH1, CDH13, CDKNIC, CFTR, COX2, DAPK1, DRG1, DRM, EDNRB, FADD, GALC, GSTP1, HNF3B, HPP1, HTERT, ICAM1, ITGA4, LAMA3, LITAF, MAGEA1, MDR1, MGMT, MINT1, MINT2, MT1GMT, MINT1, MINT2, MT1A, MTSS1, MYOD1, OCLN, p14ARF, p16INK4a, PTCHD2, RASSF1A, RPRM, RUNX3, SALL3, SERPINB5, SLC29A1, STAT1, TIMP3, TMS1, TNFRSF10A, TNFRSFIOC, TNFRSFIOD, TNFRSF21, and WWOX;

(b) providing a criteria for determining the methylation state of one or more genes to detect urinogenital cancer (e.g. bladder cancer) in the subject (specifically and sensitively).

The term “means for measuring methylation pattern of one or more genes in the urine sediments” includes any substantial technical measures, instruments, devices, and reagents that may be useful to measuring methylation pattern of one or more genes in the urine sediments. The specific means depend on the method used.

Since one preferred method of detecting the methylation state of a panel of genes is MSP and/or QMSP. The reagents included in the MSP and/or QMSP kits of this invention are apparent to the skilled in the art: reagents and materials for DNA isolation, polymerase for PCR reaction (such as Taq polymerase), sodium bisulfite, MSP/QMSP specific buffers and the corresponding primers, etc. All the related reagents (primers, among others) are included in the scope of the present invention. Primers comprise DNA, RNA, and synthetic equivalents thereof, depending on the amplification technique employed. For example, a pair of short single-stranded primers are used in standard PCR, and the two primers are localized to both sides of the target gene to be amplified (including CpG sequence, the complementation to CpG is directed to methylated region, and the complementation to TpG is directed to unmethylated gene region). The nucleic acid amplification techniques are well-known to the skilled in the art.

The present invention provided, for example, a list of verified gene primers (Table 2). However, the scope of the invention is not limited to these examples.

The present invention may also comprises methylation information of corresponding genes in urine sediments (or tissues) obtained from normal and/or non-cancerous subject.

The invention will be further understood with reference to the following examples. It should be noted that all these examples are for purpose of illustration only rather than for limitation of the scope of the invention. Unless otherwise indicated, all the techniques therein are obvious to those having basic knowledge in molecular biochemistry and relevant fields.

### EXAMPLES

### Methods

Collection of Tissues and Urine Sediments, and DNA Isolation.

With the informed consent of all patients and approval of the ethics committee, 15 samples of bladder cancer tissues were collected in Guangxi Province, China. Three normal bladder tissues were obtained from healthy organ donator. The void morning urine samples were also collected from the bladder cancer patients, diagnosed by the existing methods and standards, known in the clinical arena, at Guangxi Hospital (40) and Zhongshan Hospital, Shanghai, China (92). 79 post-surgical urine samples were also obtained at Zhongshan Hospital, Shanghai, China. The control group included 23 patients with non-cancerous urinogenital diseases (cystitis glandularis: 8, prostatic hyperplasia: 4, vesical calculus: 3, renal calculus: 5, and adrenal nodule: 3), 6 with neurological disease, and 7 healthy volunteers. The urine cytological analysis, and the tumor-node-metastasis (TNM) staging and classification are indicators according to the WHO classification and American Joint Committee on Cancer guidelines.

Bisulfite Treatment and Methylation-Specific PCR Analysis

Primer pairs for PCR detection of 59 methylated and unmethylated alleles were 1, directly from the published information, or 2. designed with software for identification of the CpG islands (http://www.ebi.ac.uk/emboss/cpgplot/index.html) and the primer design software (http://micro-gen.ouhsc.edu/cgi-bin/primer3_www.cgi) (Table 2).

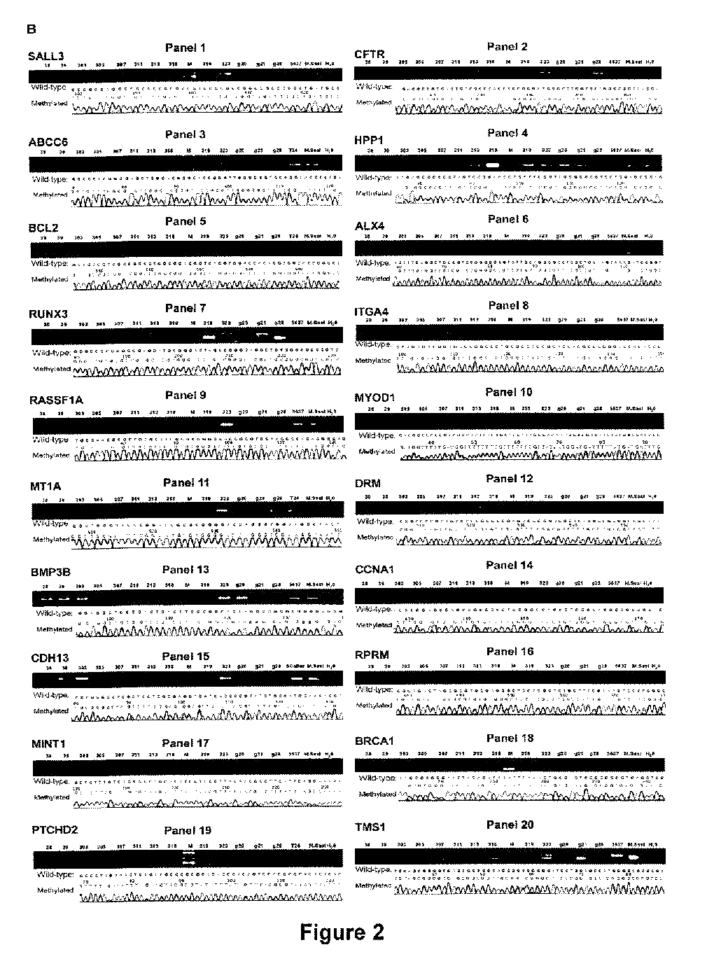

Desalting the DNA samples treated by bisulfite was carried out by a home-made agarose based gel filtration system[31, 32]. The PCR products were cloned and verified by sequencing (FIG. 2 shows 20 genes as examples). The DNA, in vitro methylated by M.Sss I, from normal liver tissues were used as a positive control.

Statistics

The significance analysis of the relation between methylation state of genes and each clinical pathological parameter was carried out by z relevant software (http://www.Rproject.org). The significance of methylation state of each gene as a bladder cancer specific marker is presented as 95% confidence interval (R package Hmisc http://cran.r-project.org/src/contrib/Descriptions/Hmisc.html). The significance of the methylation frequency of each gene in urine sediments from patients with bladder cancer (132 cases) in comparison with that from patients with non-cancerous urinogenital diseases (23 cases) is determined by 2×2 fisher exact test. The receiver operating characteristics (ROC) of both specificity and sensitivity of the gene sets useful in bladder cancer detection were calculated and plotted.

### Results

### Identification of Genes in a Bladder Cancer-Specific Methylation State

The 59 test genes (table 2) include: 1, those having been investigated in bladder cancer or other types of urinogenital tumors previously, such as CDKN2A, ARF, MGMT, GSTP1, BCL2, DAPK, and HTERT, 2, those being hypermethylated in other types of tumors according to our work [31-43], and 3, those being suggested functionally relate to carcinogenesis by bioinformatics analysis. FIG. 2A shows the methylation states of 11 diagnostically valuable genes in three established bladder cancer cell lines, and the verification of sequence analysis of the methylated and unmethylated target sequences thereof. FIG. 2B shows the MSP data of 20 diagnostically valuable genes and typical results from sequencing confirmation.

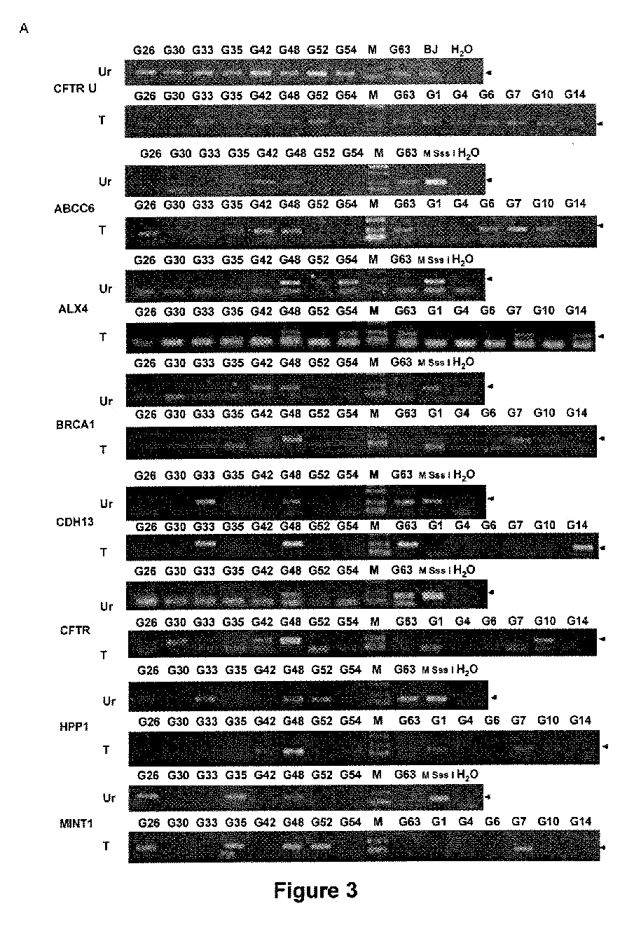

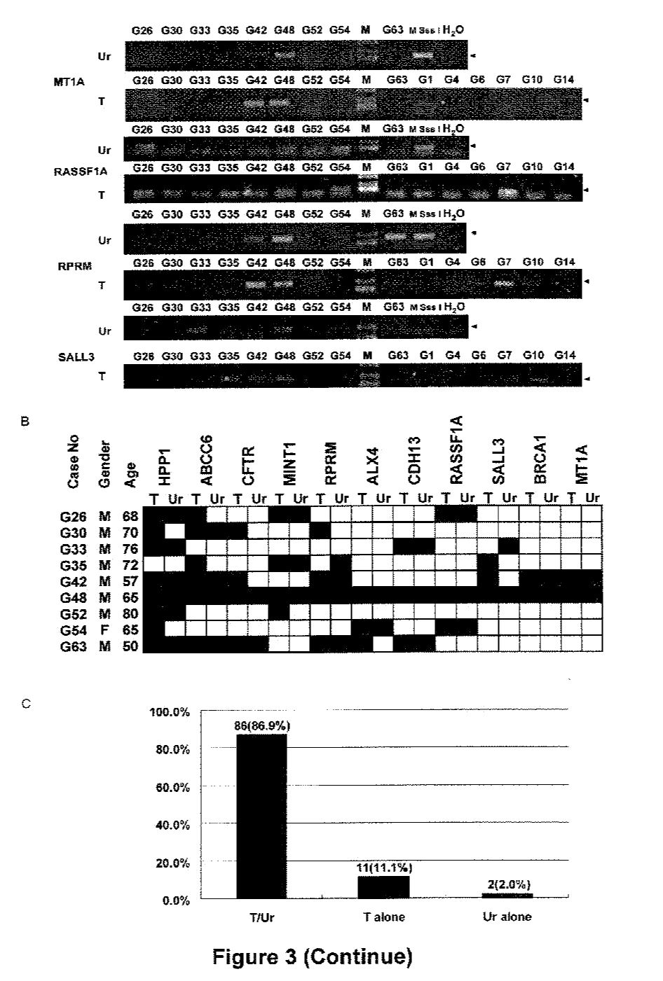

Given that the established bladder cancer cell lines are likely to contain deficiencies of clinical bladder cancer at the genetic and epigenetic level, we initially carried out MSP profiling of 59 genes on 3 bladder cancer cell lines: T24 (ATCC number: HTB-4), SCaBER (HTB-3), and 5637 (9). 41 genes were found hypermethylated, at least, in one allele of one cell line (Table 3). Although FADD, LITAF, MGMT and TNFRSF21 are homozygously unmethylated, their hypermethylation states are reported to relate to bladder cancer [44,45]. The following 14 genes have been eliminated in the initial screening: APC, BCAR3, BNIP3, CBR1, CBR3, COX2, DRG1, HNF3B, MDR1, MTSS1, SLC29A1, TIMP3, TNFRFIOA, and VVWOX. In the urine sediments of 11 patients, 21 genes were hypermethylated in 1 to 10 patients (9% to 90%), but not in 3 patients with cystitis glandularis. It is implicated that the hypermethylation states of these genes relate to various degrees of bladder cancer-specificity. The characteristic promoter unmethylation of the MAGEA1 gene and concomitant activation of transcription are frequently found in cancer. However, in the present study of bladder cancer, this phenomenon occurs scarcely (Table 3), the releant study is terminated thereby. This was also the reason to exclude LAMA3, ICAM1, and GALC. We further analyzed 15 cancer tissues and 3 normal bladder tissues for the DNA methylation state of 32 genes. Although 28 genes were unmethylated in the 3 normal bladder tissues, 19 genes among which were hypermethylated in 1-12/15(6.7% to 73.3%) bladder cancer tissues, indicating various degrees of bladder cancer specificity. The other genes: PTCHD2, BRCA1, CDH13, TMS1, CDH1, p14ARF, p16INK4a, FADD, LITAF, MGMT, and TNFRSF2, are also unmethylated. To determine the association of DNA methylation patterns between tumor tissues and cells from urine sediments, we have carried out MSP-profiling of 9 pairs of samples (FIG. 3). Among 99 methylation events, 86 (87%) were shared by the tumor tissues and corresponding urine sediments, 11 (11%) were unique to tumor tissues, and 2 (2%) were unique to urine sediments. The inconsistency is low, but is still 13%. Therefore, the genes only methylated in one kind of samples were included for a further study: BRCA1 and CDH13 (only hypermethylated in tumor tissues), and PTCHD2 (only hypermethylated in urine sediments). TMS1 was also included for the further analysis as it was reported as one of the most informative markers for prostate cancer in USA[44], however, it is not reported to date that its methylation state relates to bladder cancer.

Methylation States of 21 Genes in DNA of Urine Sediments from Bladder Cancer Patients and Non-Bladder Cancer Control Group

The test samples are from bladder cancer cohort (132) and 3 control groups, namely, 1), neurological disease (6), 2), healthy volunteers (7), and 3), non-cancerous urinogenital disease (23), including cystitis glandularis: 8, prostatic hyperplasia: 4, vesical calculus: 3, renal calculus: 5 and adrenal nodule: 3. The average age of the bladder cancer cohort was 63.4 (34-88), which matched well to that for the non-cancerous urinogenital disease cohort, i.e. 55.7 (16-83) and the neurological diseases cohort, i.e. 64.1 (46-78).

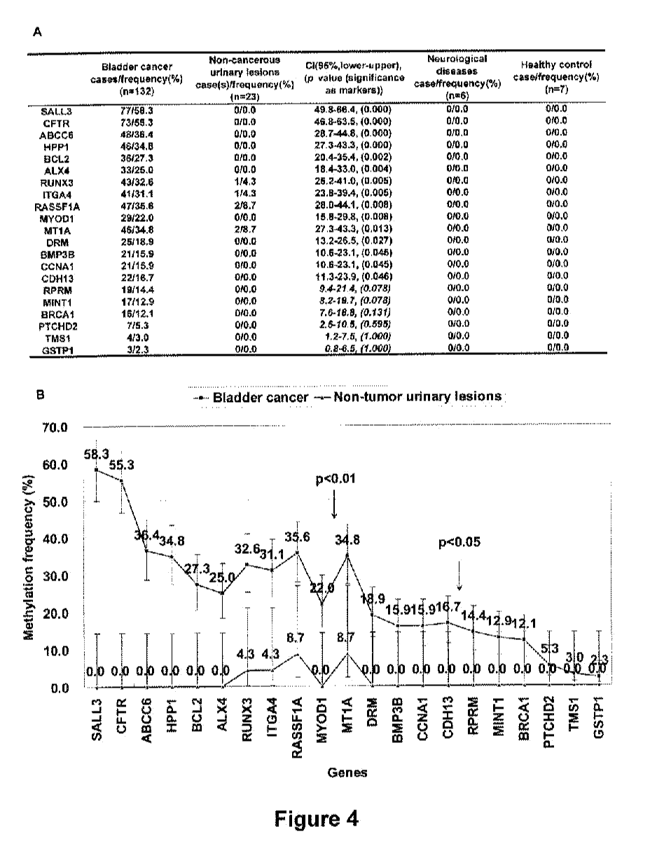

The 21 genes were unmethylated in the urine sediments from healthy volunteers and patients with the neurological disease. However, 6 hypermethylation events were recorded in four genes: RASSF1a (2/23), MT1A (2/23), RUNX3 (1/23) and ITGA4 (1/23) (FIG. 4A), which involved 3 patients in non-cancerous urinogenital disease cohort (including 2 patients with prostatic hyperplasia (84, and 64 years old) and 1 patient with vesical calculus (54 years old)). The influence of the “false positive” results on the criteria for bladder cancer detection was taken into consideration by corresponding statistic analysis (FIGS. 4A and 4B). Four relevant genes, with the highest frequency of DNA hypermethylation in urine sediments from bladder cancer patients and in unmethylated states in control cohorts, were identified: SALL3 (58.3%, CI (Confidence Interval): 95%: 49.8%-66.4%), CFTR (55.3% CI: 95%: 46.8%-63.4%), ABCC6 (36.4% CI 95%: 28.7%-44.8%), and HPP1 (34.8% CI 95%: 27.3%-43.3%). The rest 6 genes with a p value of <0.01 were BCL2 (27.3% CI 95%: 20.4%-35.4%), ALX4 (25% CI 95%: 18.4%-33%), RUNX3 (32.6% CI 95%: 25.2%-41%), ITGA4 (31.1%, CI 95%: 23.8%-39.4%), RASSF1A (35.6% CI 95%: 28%-44.1%), and MYOD1 (22% CI 95%: 15.8%-29.8%). The genes with a p value of <0.05 were MT1A (34.8% CI 95%: 27.3%-43.3%), DRM (18.9% CI 95%: 13.2%-26.5%), BMP3B (15.9% CI 95%: 10.6%-23.1%), CCNA1 (15.9% CI 95%: 10.6%-23.1%), and CDH13 (16.7%, CI 95%: 11.3-23.9%). The genes hypermethylated in more than 12.1% of bladder cancer cases are RPRM, MINT1, and BRCA1. These genes may have certain_values in diagnosing bladder cancer. This observation contradicts the previous report [44], both TMS1 (P=1) and GSTP1 (p=1) were found hypermethylated only in 2 bladder cancer patients (5.3% (2/132)). By taking the hypermethylated state of any gene in the 11 genes as an indicator for bladder cancer, 121 of the 132 bladder cancer patients were positive (92%), wherein 6 of 8 are in stage 0a (sensitivity: 75%), 60 of 68 are in stage I (88.2%), 49 of 50 are in stage II (98.2%), 4 of 4 are in stage III (100%), and 2 of 2 are in stage IV (100%)(Table 5). As compared to the results from the urine cytological analysis (detected 1 case in stage I, and 2 cases in stage II, but missed 17 cases, including 4 cases in stage 0a), 19 of 20 cases, except for one case (among four) in stage 0a, were detected by the present analysis, indicating the much higher sensitivity of the present method than the urine cytological analysis.

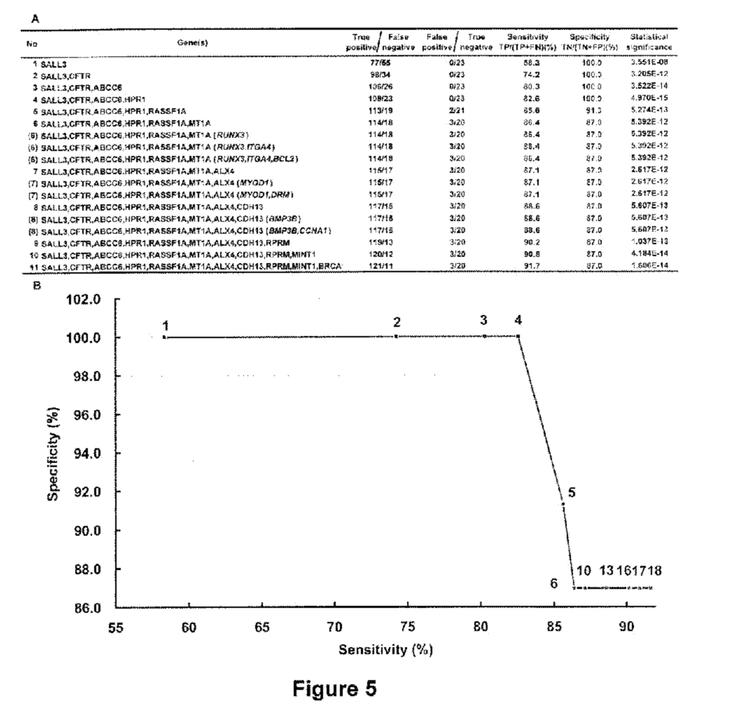

We failed to find the substantial association of the DNA methylation of genes with cancer staging (Table 5) by the statistic test. Comparing with the DNA methylation state in the urine sediments from 79 post-surgical patients, we found that the methylation incidence of MYOD1 and MINT1 turned from 22.2% and 12.9% before surgery to 0% after surgery, respectively, the incidence of methylation of other genes are also substantially reduced (P<0.005)(Table 6). The methylated genes remained in urine sediment were likely caused by the incomplete removal of tumor by the surgical procedure. Therefore, analysis of the DNA methylation pattern in urine sediments from pre- or post-surgical patients can be effective to assess the surgical quality. Additionally, no significance difference was found in the DNA methylation patterns between the primary and recurrent cases of bladder cancer (p>0.05) (Table 7). The methylation of a single gene (SALL3) can be used to detect at most 58.3% of the bladder cancer cases, and detection of multiple genes may improve the detection rate and specificity for bladder cancer. Hypermethylation of 10 genes results in extremely high tumor-specificity (p<0.01), and hypermethylation of 5 additional genes also results in substantial tumor-specificity (p<0.05 (FIGS. 4A and 4B)). The low frequency of methylation was found in 3 genes in the non-cancerous urinogenital disease control cohort, which has influence on the specificity of these genes as indicator of bladder cancer. “True positive” (TP) was defined as a bladder cancer sample having at least one gene methylated, while “False negative” (FN) was defined as a bladder cancer sample having no gene methylated. “False positive” (FP) was defined as the non-cancerous urinogenital disease sample having at least one gene methylated, while “True negative” (TN) was defined as the non-cancerous urinogenital disease sample having no gene methylated. Both “Sensitivity”=TP/(TP+FN) (%, Column 4 in FIG. 5A) and “specificity”=TN/(TN+FP) (%, Column 5, Table 5A) of each gene were calculated. The receiver operating characteristics (ROC) of both specificity and sensitivity for sets of 2-11 genes were shown in FIG. 5.

None of the following four genes: SALL4, CFTR, ABCC6, and HPP1 were false positive in three control groups, the specificity for them, alone or in combination, to detect bladder cancer should be 100% (FIG. 4). The sensitivity was: 58% (77/132) for SALL3 alone, 74.2% (98/132) for SALL3 and CFTR, 80.3% (106/132) for SALL3, CFTR, and ABCC6, and 82.6% (109/132) for SALL3, CFTR, ABCC6, and HPR1(Column 4 and 5, FIG. 5A).

| | Bladder | Non-cancerous |
|---|---|---|
| | cancer (123) | control (23) |
| Methylated | TP(121) | FP(3) |
| Unmethylated | FN(12) | TP(20) |

The first column indicates the gene sets. The genes in bracket were considered redundant as inclusion thereof did not improve the sensitivity of the set. The second column indicates the number of the true positive (TP=the bladder cancer sample having at least one gene methylated) and false negative (FN=the bladder cancer sample having no gene methylated) events. The third column indicates the number of the false positive (FP=the non-cancerous urinogenital disease sample having at least one gene methylated) and true negative (TN=the non-cancerous urinogenital disease sample having no gene methylated) events. Both Sensitivity=TP/(TP+FN) (%, Column 4) and specificity=TN/(TN+FP) (%, Column 5) of each gene sets were calculated and plotted in FIG. 5A.

The hypermethylated RASSFIA gene was found in 2 of 23 cases in the non-cancerous urinogenital disease group (2 false positive events and 21 true negative events, Column 3, FIG. 4A). Therefore, its inclusion in a 5 gene set improved the sensitivity to 85.6%, with a compromised specificity: 91.3% (Column 4 and 5, FIG. 5A). The six gene set with MT1A had an improved sensitivity: 86.4% and a moderately reduced specificity: 87%, as MT1A was also methylated in another sample of the non-cancerous urinogenital disease group (the accumulated false positive events: 3, and true negative events: 20, column 3, FIG. 5A). Given that further addition of gene RUNX, ITGA4, or BCL2 did not improve the sensitivity of the detection, they were not taken as valuable markers. The sensitivity of a 7 gene set with additional ALX4 is 87.1%, that of a 8 gene set with additional CDH13 is 88.6%, that of a 9 gene set with additional RPRM is 90.2%, that of a 10 gene set with additional MINT is 90.9%, and that of a 11 gene set with additional BRCA1 is 91.7, however, the specificity remained 87%.

Although the aforementioned description relates to particular examples, the spirit and scope of the present invention, and modifications of these information and practical forms according to the established principles are apparent to those skilled in the art. Therefore, such possible modifications should be within the scope of the following claims.

TABLE 1
Molecular Biomarkers for Cancer Detection

| | Genetic | Epigenetic | | |
|---|---|---|---|---|
| | Mutation, | DNA | Expressional | |
| | SNP, LOH | methylation | mRNA | Protein |
| Stability | High | High | Low | Low |
| PCRable | Yes | Yes | Yes | No |
| Target/gene | Multiple | Single | NA | NA |
| Nature | Quantita- | Qualita- | Quantita- | Quantita- |
| | tive | tive | tive | tive |
| Sample purity | Essential | Non- | Essential | Essential |
| | | essential | | |
| Fluctuation | No | No | Yes | Yes |
| Tumor type | Low | High | Low | Low |
| specificity | | | | |

NA, not applicable; multiple/single: one (single) or more than one (multiple) targets need to be analyzed; fluctuation, whether the amout of the biomarker changes with the fluctuation of non-cancerous factors (biological clock, physiological, or pathological factors); SNP: single nucleotide polymorphism; LOH: loss of heterozygosity.

TABLE 2
Primer list for the MSP-profiling of the promoter CpG islands of the genes

| | | | | | Location of | |
|---|---|---|---|---|---|---|
| | | | | | product | |
| | | | | | fragment | |
| | | | | | relative to | |
| Or- | | | | | transcription | |
| der | | GenBank | | | initiation | Size |
| No. | Gene Name | No. | Sense 5′-3′ | Antisense 5′-3′ | site | (bp) |
| 1 | ABCC13M | NT_011512 | GCGGGCGGTTTTTATTAG | CAAAAACTCGTCCGTCCA | +314~+478 | 165 |
| | ABCC13U | | TGGGTTTGTGGGGTGTT | ACAAAAACTCATCCATCCACAT | +332~+479 | 148 |
| 2 | ABCC6M | NT_010393 | GGCGTTCGGGGAGTT | CGACCTCGACCCGATAAT | −436~−190 | 247 |
| | ABCC6U | | AGGTGTTTGGGGAGTTGG | TCTCAACCTCAACCCAATAATC | −437~−194 | 244 |
| 3 | ABCC8M | NT_009237 | GACGTGCGGTATTACGTTG | ACAAAAACGCGACAAACG | +72~+254 | 183 |
| | ABCC8U | | AGGATGGGGAAGGTGATG | AAAACAAAAACACAACAAACACAC | +75~+282 | 208 |
| 4 | ALX4M | NT_009237 | GAGTTTGAGGTTGTCGTTCG | AACCCGTTACGACGCTAAAC | +311~+539 | 229 |
| | ALX4U | | TTGTTTGGGGGTGTTTTG | AAACCAAACCCATTACAACACT | +307~+527 | 221 |
| 5 | APCM | NT_034772 | TATTGCGGAGTGCGGGTC | TCGACGAACTCCCGACGA | −163~−66 | 98 |
| | APCU | | GTGTTTTATTGTGGAGTGTGG | CCAATCAACAAACTCCCAACAA | −169~−62 | 108 |
| | | | GTT | | | |
| 6 | BCAR3M | NT_028050 | GCGTTTCGGGAGGAATAG | ACTACGAAACGCACCGACT | −137~+103 | 241 |
| | BCAR3U | | TGGGTGTGTGGTGGAGAT | CTACAAAACACACCAACTAAACACA | −136~+71 | 208 |
| 7 | BCL2M | NT_025028 | GAAGTCGTCGTCGGTTTG | CCCGCACCGAACATC | +276~+458 | 183 |
| | BCL2U | | TTGTTGTTGGTTTGGTGGA | CCCACACCAAACATCTTCTC | +276~+454 | 179 |
| 8 | BMP3BM | NT_030772 | GCGGTAAAGGGTCGAAGT | AACTCGAACCGCCGATA | +65~+460 | 196 |
| | BMP3BU | | TGAGGGTGGTAAAGGGTTG | AAAAACTCAAACCACCAATACC | +267~+460 | 194 |
| 9 | BNIP3M | NT_024040 | TCGTTCGGTTTCGTTTTG | ACGCTCCGTTCTACGACA | −49~+144 | 194 |
| | BNIP3U | | GTTGTAGATTTGTTTGGTTTTG | ACATCCCAAACACTCCATTCT | −58~+153 | 212 |
| | | | TTT | | | |
| 10 | BRCA1M | L78833 | GGTTAATTTAGAGTTTCGAGAG | TCAACGAACTCACGCCGCGCAATCG | −320~−138 | 183 |
| | | | ACG | | | |
| | BRCA1U | | GGTTAATTTAGAGTTTTGAGAG | TCAACAAACTCACACCACACAATCA | −320~−138 | 183 |
| | | | ATG | | | |
| 11 | BRCA2M | NT_024524 | GCGGAGATTGCGTTATTG | CCGAACCCGTTTCCTTAC | −682~−519 | 164 |
| | BRCA2U | | TGGAGGTGGAAGTTGTGG | CTCCAAACCCATTTCCTTACT | −703~−517 | 187 |
| 12 | CBR1M | NT_086913 | TCGTATTTGGCGAGGT | AAACCCCGCAACGTATTC | −126~+36 | 163 |
| | CBR1U | | TTGGTGGGGAGGGGTA | AAACCCCACAACATATTC | −108~+36 | 145 |
| 13 | CBR3M | NT_086913 | CGTAGATTATTTCGCGGTTTAG | GAACCGAACTTCGAACCAC | −260~−14 | 247 |
| | CBR3U | | GGGTGTAGTGTGGGTAGGG | AAACCAAACTTCAAACCACCT | −223~−14 | 210 |
| 14 | CCNA1M | AF124143 | TCGTCGCGTTTTAGTCGT | ACCCGTTCTCCCAACAAC | −755~−550 | 206 |
| | CCNA1U | | GGGTAGTTTTGTTGTGTTTTAG | AACCACTAACAACCCCCTCT | −762~−565 | 198 |
| | | | TTG | | | |
| 15 | CDH1M | L34545 | GTGGGCGGGTCGTTAGTTTC | CTCACAAATACTTTACAATTCCGACG | −265 to −93 | 172 |
| | CDH1U | | GGTGGGTGGGTTGTTAGTTTTGT | AACTCACAAATCTTTACAATTCCAAC | −266 to −93 | 172 |
| 16 | CDH13M | AB001090 | TCGCGGGGTTCGTTTTTGC | GACGTTTTCATTCATACACGCG | −267~−24 | 244 |
| | CDH13U | | TTGTGGGGTTTGTTTTTTGT | AACTTTTCATTCATACACACA | −267~−24 | 244 |
| 17 | CDKN1CM | NT_009237 | GGTTCGGTTTTCGCGTAT | AAAACGAACGTCGCGATA | −354~−159 | 196 |
| | CDKN1CU | | TTTGTTGTGGTTTGGTTTTTG | AACAAACATCACAATATCACATTACC | −344~−148 | 197 |
| 18 | CFTRM | N7_007933 | AGAGGTCGCGATTGTCGTT | CGACTTTCTCCACCCACTACG | −316~−114 | 203 |
| | CFTRU | | TTAAAGAGAGGTTGTGATTGTT | TCCTTCACTCCCTCACCA | −322~−174 | 149 |
| | | | GTT | | | |
| 19 | COX2M | NT_004487 | GTTCGTCGTTGCGATGTT | CCAAACTCTTTCCCAAATCA | +122~+324 | 203 |
| | COX2U | | TTGTTTGTTGTTGTGATGTTTG | TCCAAACTCTTTCCCAAATC | +120~+325 | 206 |
| 20 | DAPK1M | NT_023935 | TCGGTAATTCGTAGCGGTAG | TACTCACCCGAACGCCTA | +57~+234 | 178 |
| | DAPK1U | | GGGATTTGGTAATTTGTAGTGG | CCTAACTACTCACCCAAACACCT | +52~+240 | 189 |
| 21 | DRG1M | NT_011520 | GGTGCGGAGTATGAGTCG | CCGCGAACCAATACGATA | −335~−132 | 204 |
| | DRG1U | | GTGAGGAATAGGGGTGTGG | CCCACAAACCAATACAATATCAT | −347~−131 | 217 |
| 22 | DRMM | NT_010194 | TCGGTTTCGTTGATTTCG | AAACTACCGCGCGTAAAAC | −42~+155 | 198 |
| | DRMU | | TTGAGTTTTGGTGGTTTTGG | AAACTACCACACATAAAAC | −22~+155 | 178 |
| 23 | ENDRBM | NT_024524 | TAGGGCGCGTTCGTATAG | CCACTAACGCGCAAACTT | −119~+103 | 223 |
| | ENDRBU | | TGTGTTTGTATAGATTTGGAG | TTCCCACTAACACACAAACTTAAA | −116~+104 | 221 |
| | | | GTG | | | |
| 24 | FADDM | NT_033927 | CGTGACGTTCGGGTTG | CCTACGCCCGACGTATC | −169~+19 | 189 |
| | FADDU | | TGGATTTGGTAGAGGTGTGATT | TACACCTACACCCAACATATCATC | −96~+24 | 121 |
| 25 | GALCM | NT_026437 | GGTGACGTCGGAAGAGAAG | CCGCCACGATAAATACGA | +93~+289 | 197 |
| | GALCU | | TTATTAGGTGATGTTGGAAGAG | AAAAACAAATCCCATCACCA | +67~+306 | 220 |
| | | | AAG | | | |
| 26 | GSTP1M | NT_033903 | GCGATTTCGGGGATTTTA | ACGACGACGAAACTCCAA | −183~+15 | 199 |
| | GSTP1U | | GTTGGGGATTTGGGAAAG | TATAAAAATAATCCCACCCCACT | −230~−28 | 203 |
| 27 | HNF3BM | NT_011387 | CGTTCGTTGTTGTTTTTGC | AACCGTCGACCGCTACTAA | +13~+199 | 187 |
| | HNF3BU | | GGGAGAAGTGTGGGGTGT | CCCAACCATCAACCACTACTAA | +13~+139 | 127 |
| 28 | HPP1M | AF242221 | AAGAGGGGCGTTAGTTCG | CGCTCGCAAACGCTAA | −320~−163 | 158 |
| | HPP1U | | ATGTGTGGAAGAGGGGTGT | CACTCACAAACACTAACCCAAA | −328~−163 | 166 |
| 29 | HTERTNM | NT_006576 | GCGTCGCGAGGAGAG | AATTCGCGAACACAAACG | −205~+4 | 210 |
| | HTERTNU | | GGGGTTGTGGAAAGGAAG | AACCACACTTCCCACATAACA | −179~−11 | 169 |
| 30 | ICAM1M | NT_011295 | TAGCGCGGTGTAGATCGT | CGAACTAACAAAATACCCGAAC | −284~−101 | 184 |
| | ICAM1U | | TTGGGAAATGGGAGGTG | TCCAAACTAACAAAATACCCAAAC | −248~−99 | 150 |
| 31 | ITGA4M | NT_005403 | GACGCGAGTTTTGCGTAG | TAAAATACCGCGCACTCG | +779~+978 | 200 |
| | ITGA4U | | GGGAGGTTTGGGTTAGGAT | CAACCTAAAATACCACACACTCAC | +763~+983 | 221 |
| 32 | PTCHD2M | NT_021937 | TTTCGCGGTCGTTTTAGA | CCGCCCACGTACGTATAA | +1037~+1237 | 201 |
| | PTCHD2U | | TGGATAGTGTTTTGTGGTTGTTT | CCACCCACATACATATAAACCAT | +1028~+1237 | 210 |
| 33 | LAM3M | NT_010966 | TTCGTTCGCGAAGTTTGT | TAAACGACGCCGAAACC | −217~−29 | 189 |
| | LAM3U | | TGTGTTTTGTGTGGGAGAGA | AAACAACACCAAAACCACTCC | −197~−30 | 168 |
| 34 | LITAFM | NT_010393 | CGGTCGGGTTTTTACGTT | ACCTCCCGACTCGACAA | −528~−314 | 215 |
| | LITAFU | | GGGAGGTTGGATTTTGTTTT | CAAACCTCCCAACTCAACAA | −528~−293 | 236 |
| 35 | MAGEA1M | NT_011726 | GTTCGGTCGAAGGAATTTGA | CCACAACCCTCCCTCTTAAA | +7~+328 | 322 |
| | MAGEA1U | | GTTTGGTTGAAGGAATTTGA | ACCCACAACCCTCCCTCTTA | +7~+330 | 324 |
| 36 | MDR1M | NT_007933 | TTGGGGGTTTGGTAGCGC | CTCTCTAAACCCGCGAACGAT | +112864~+112749 | 115 |
| | MDR1U | | GTTGGGGGTTTGGTAGTGT | ACTCTCTAAACCCACAAACAAT | +112864~+112748 | 117 |
| 37 | MGMTM | NT_008818 | AGCGTCGTTGTTTTGTGC | CGCTTTCAAAACCACTCG | −439~−254 | 186 |
| | MGMTU | | TTGGTAGTGTTGTTGTTTTGTGT | CATCCTACAACCCCCACA | −457~−249 | 209 |
| 38 | MINT2M | AF135502 | TGTTGGTGGATTTTGGATTT | AACAACAATTCCATACACCTTTCT | +446~+551 | 106 |
| | MINT2U | | AGTTCGTTGGCGGATTTT | CCCGAAATAATAACGACGATT | +442~+562 | 121 |
| 39 | MINT1M | AF135501 | TTCGAAGCGTTTGTTTGG | CGCCTAACCTAACGCACA | +169~+328 | 160 |
| | MINT1U | | TATTTTTGAAGTGTTTGTTTGG | TCCCTCTCCCCTCTAAACTTC | +165~+366 | 202 |
| | | | TGT | | | |
| 40 | MT1AM | K01383 | TAAGGTTGGGTTTTCGGAAC | AAATACGAACCACGAAACCA | −421~−258 | 164 |
| | MT1AU | | TAAGGTTGGGTTTTTGGAAT | CTCCCCTAAATACAAACCACA | −421~−251 | 171 |
| 41 | MTSS1M | NT_008046 | TGATTTCGGTCGGGAGT | AAATACAACGCGCTCGAA | +501~+697 | 197 |
| | MTSS1U | | GGTGATATTTTGGTTGGGAGT | AAATACAACACACTCAAAAACCTCT | +508~+701 | 194 |
| 42 | MYOD1M | AF027148 | GACGGTTTTCGACGGTTT | GCCCGAAACCGAATACAC | +210~+393 | 184 |
| | MTOD1U | | ATTTGATGGTTTTTGATGGTTT | CACACACATACTCATCCTCACA | +206~+418 | 213 |
| 43 | OCLNM | NT—006713 | TGCGTTCGTTAGGTGAGC | CGAATCCCAACTCGAAAACG | +537~+762 | 216 |
| | OCLNU | | GTTAGGTGTGTTTGTTAGGTG | CACACCTCTCTAATTCCCACA | +531~+771 | 241 |
| | | | AGT | | | |
| 44 | p14^{ARF}M | L41934 | GTCGAGTTCGGTTTTGGAGG | AAAACCACAACGACGAACG | 95 TO 255 | 160 |
| | p14^{ARF}U | | TGAGTTTGGTTTTGGAGGTGG | AACCACAACAACAAACACCCCT | 97 TO 262 | 165 |
| 45 | p61^{INK4a}M | NM_000077 | TTATTAGAGGGTGGGGCGGAT | ACCCCGAACCGCGACCGTAA | −80 to 69 | 149 |
| | | | CGC | | | |
| | p61^{INK4a}U | | TTATTAGAGGGTGGGGTGGAT | CAACCCCAAACCACAACCATAA | −80 to 71 | 151 |
| | | | TGT | | | |
| 46 | RASSF1AM | XM_040961 | GTGTTAACGCGTTGCGTATC | AACCCCGCGAACTAAAAACGA | +82~+176 | 95 |
| | RASSF1AU | | TTTGGTTGGAGTGTGTTAATGTG | CAAACCCCACAAACTAAAAACAA | +70~+178 | 109 |
| 47 | RPRMM | NT_005403 | TGAGCGTTTATTCGTAGATTAGC | GAACGAACGCCGAAAAC | +14~+184 | 171 |
| | RPRMU | | GTGGTGGTGTTGGAGGAA | TCAAACAAACACCAAAAACAAAC | +18~+209 | 192 |
| 48 | RUNX3M | NT_004610 | GAGGGGCGGTCGTACGCGGG | AAAACGACCGACGCGAACGCCTCC | −259~−44 | 216 |
| | RUNX3U | | GAGGGGTGGTTGTATGTGGG | AAAACAACCAACACAAACACCTCC | −259~−44 | 216 |
| 49 | SALL3M | NT_010879 | GTTCGCGTAGTCGTCGTC | TACTCGAAAACCCCGTCA | −123~+79 | 203 |
| | SALL3U | | GTGGTTTGTGTAGTTGTTGTT | CCCAACCCTCACCATACTC | −126~+93 | 220 |
| | | | GTT | | | |
| 50 | SERPINB5M | NT_025028 | TTTGCGTGGGTCGAGA | GCCTCGACGACACTCC | −219~−29 | 191 |
| | SERPINB5U | | TTTTGTGTGGGTTGAGAGG | CACCCCACCCCACCT | −220~−18 | 203 |
| 51 | SLC29A1M | NT_007592 | AAGGCGTCGGTCGTTAGT | TATAAACCGCCGAACGAA | −178~−18 | 161 |
| | SLC29A1U | | TGGGTGTTTAAAGGTGTTGG | ACCAATATAAACCACCAAACAAA | −188~−13 | 176 |
| 52 | STAT1M | NT_005403 | GTCGTTCGGTGATTGGTG | AACGAAAACGCGACGATA | −28~+166 | 195 |
| | STAT1U | | TGTTTAATTGGTTGAGTGTGGA | AAACTAAACAAAAACACAACAATACAA | −50~+172 | 223 |
| 53 | TMS1M | NT_010393 | TTGTAGCGGGGTGAGCGGC | AACGTCCATAAACAACAACGCG | +197~+387 | 191 |
| | TMS1U | | GGTTGTAGTGGGGTGAGTGGT | CAAAACATCCATAAACAACAACACA | +195~+390 | 196 |
| 54 | TNFRSF10AM | NT_023666 | GTTTTTCGGTCGGGAGTT | ACTCGCCCGATAATAACGA | −321~−160 | 162 |
| | TNFRSF10AU | | TGTTTGGTGGATGGATGG | ACTAAATCACTCACCCAATAATAACAA | −321~−220 | 102 |
| 55 | TNFRSF10CM | NT_023666 | AGCGTTTCGGTCGTTTG | TACCGTATCCCCGTCTCC | +131~+338 | 208 |
| | TNFRSF10CU | | TGGTTGAGGTAGGGTGTGAT | TACCATATCCCCATCTCCCTA | +149~+338 | 190 |
| 56 | TNFRSF10DM | NT_023666 | GAATCGCGACGATGAAGA | CACGCGCACAAACTACG | +38~+250 | 213 |
| | TNFRSF10DU | | AGAATTGTGATGATGAAGATG | AACCTTTACACACACACAAACTACA | +38~+257 | 220 |
| | | | ATG | | | |
| 57 | TNFRSF21M | NT_007592 | TTGTTTAGCGTCGTATTTATCGT | TCCTCAACCGCTATCGAA | +169~+390 | 222 |
| | TNFRSF21U | | TTTTTGGGTTGGGAGTTTATT | TAATTCTCCTCAACCACTATCAAAA | +170~+362 | 193 |
| 58 | WWOXM | NT_0140498 | GCGATATTGCGGAGATTG | CCCTATCGCCCGCTAC | −58~+99 | 158 |
| | WWOXU | | TTGTGGAGATTGGATTTTAGT | CCCTATCACCCACTACCAAAT | −52~+99 | 152 |
| | | | TTT | | | |

(SEQ ID NOS 1-236, respectively, in order of appearance.)

TABLE 3
Methylation states of the tested genes

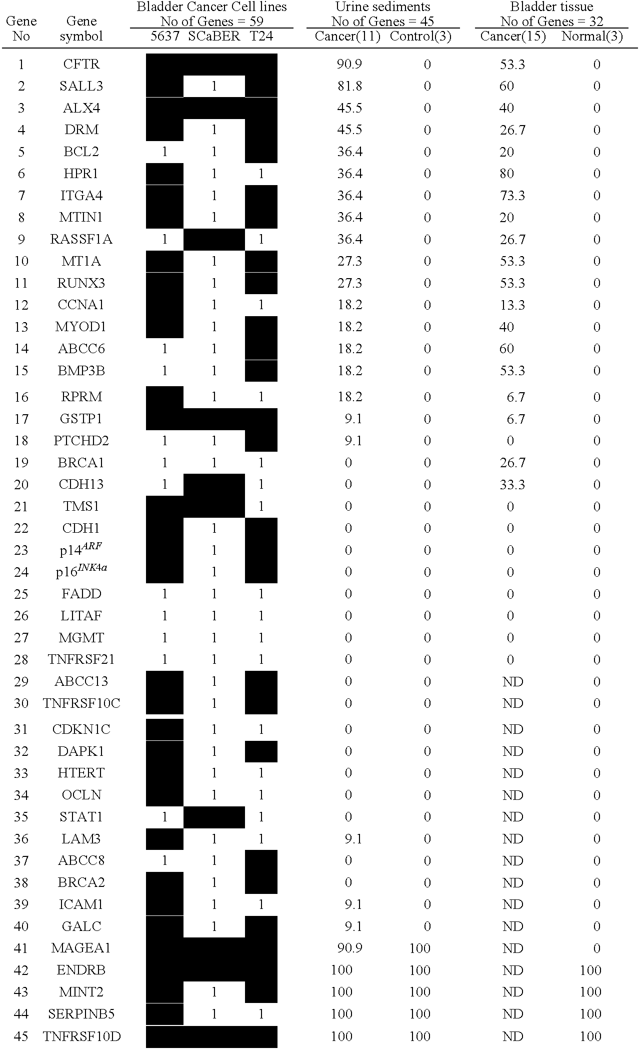

N.B., 1, the homozygously unmethylated; 2, in grey background: heterozygously methylated; and 3, in dark background: homozygously methylated. The number of tested genes is shown and the number of clinical samples is shown in brackets. The urine sediments derived from patients with cystitis glandularis are used as non-bladder cancer control. The following genes are homozygously methylated in tumor cells, thereby not shown.

TABLE 4
Clinical profile of the bladder cancer patients and controls

| | | | Non-cancerous | Neuro- | |
|---|---|---|---|---|---|
| | | Bladder | urinogenital | logical | Healthy |
| | | cancer | diseases | diseases | control |
| | | (n = 132) | (n = 23) | (n = 6) | (n = 7) |
| Gender | F | 25 | 6 | 2 | 4 |
| | M | 107 | 17 | 4 | 3 |
| Age | 19-30 | 0 | 2 | | 6 |
| | 31-40 | 5 | 2 | | 1 |
| | 41-50 | 22 | 4 | 1 | |
| | 51-60 | 24 | 7 | | |
| | 61- | 81 | 8 | 5 | |
| | Range | 34-88 | 16-83 | 46-78 | 23-34 |
| | Average | 63.4 | 55.7 | 64.1 | 25.7 |
| Stage | 0a | 8 | | | |
| | I | 68 | | | |
| | II | 50 | | | |
| | III | 4 | | | |
| | IV | 2 | | | |
| Primary | | 99 | | | |
| cases | | | | | |
| Recurrent | | 33 | | | |
| cases | | | | | |

TABLE 5
DNA methylation profiles in urine sediments from bladder cancer patients and TMN staging

| | Stage | | | | | |
|---|---|---|---|---|---|---|
| | 0a | I | II | III | IV | Total |
| | case(s)^{/} | case(s)/ | case(s)/ | case(s)/ | case(s)/ | case(s)/ |
| Gene | frequency(%) | frequency(%) | frequency(%) | frequency(%) | frequency(%) | frequency(%) |
| Symbol | (n = 8) | (n = 68) | (n = 50) | (n = 4) | (n = 2) | (n = 132) |
| SALL3 | 4/50.0 | 31/45.6 | 36/72.0 | 4/100.0 | 2/100.0 | 77/58.3 |
| CFTR | 5/62.5 | 36/52.9 | 26/52.0 | 4/100.0 | 2/100.0 | 73/55.3 |
| ABCC6 | 1/12.5 | 19/27.9 | 25/50.0 | 2/50.0 | 1/50.0 | 48/36.4 |
| HPP1 | 2/25.0 | 22/32.4 | 21/42.0 | 0/0.0 | 1/50.0 | 46/34.8 |
| BCL2 | 3/37.5 | 15/22.1 | 17/34.0 | 0/0.0 | 1/50.0 | 36/27.3 |
| ALX4 | 4/50.0 | 15/22.1 | 12/24.0 | 2/50.0 | 0/0.0 | 33/25.0 |
| RUNX3 | 3/37.5 | 17/25.0 | 22/44.0 | 1/25.0 | 0/0.0 | 43/32.6 |
| ITGA4 | 1/12.5 | 16/23.5 | 21/42.0 | 2/50.0 | 1/50.0 | 41/31.1 |
| RASSF1A | 0/0.0 | 19/27.9 | 25/50.0 | 1/25.0 | 2/100.0 | 47/35.6 |
| MYOD1 | 1/12.5 | 12/17.6 | 15/30.0 | 0/0.0 | 1/50.0 | 29/22.0 |
| MT1A | 1/12.5 | 22/32.4 | 21/42.0 | 1/25.0 | 1/50.0 | 46/34.8 |
| DRM | 0/0.0 | 15/22.1 | 9/18.0 | 1/25.0 | 0/0.0 | 25/18.9 |
| BMP3B | 0/0.0 | 9/13.2 | 11/22.0 | 1/25.0 | 0/0.0 | 21/15.9 |
| CCNA1 | 1/12.5 | 7/10.3 | 12/24.0 | 1/25.0 | 0/0.0 | 21/15.9 |
| CDH13 | 0/0.0 | 12/17.6 | 9/18.0 | 1/25.0 | 0/0.0 | 22/16.7 |
| RPRM | 1/12.5 | 9/13.2 | 7/14.0 | 2/50.0 | 0/0.0 | 19/14.4 |
| MINT1 | 2/25.0 | 6/8.8 | 7/14.0 | 1/25.0 | 1/50.0 | 17/12.9 |
| BRCA1 | 0/0.0 | 7/10.3 | 8/16.0 | 1/25.0 | 0/0.0 | 16/12.1 |
| PTCHD2 | 0/0.0 | 4/5.9 | 2/4.0 | 1/25.0 | 0/0.0 | 7/5.3 |
| TMS1 | 0/0.0 | 2/2.9 | 2/4.0 | 0/0.0 | 0/0.0 | 4/3.0 |
| GSTP1 | 0/0.0 | 2/2.9 | 1/2.0 | 0/0.0 | 0/0.0 | 3/2.3 |

TABLE 6
Methylation profiles in urine sediments from bladder
cancer patients before and after surgery

| | Pre-surgery | Post-surgery | |
|---|---|---|---|
| | case(s)/ | case(s)/ | |
| Gene | frequency(%) | frequency(%) | |
| Symbol | (n = 132) | (n = 79) | p value |
| SALL3 | 77/58.3 | 6/7.6 | 1.543E−14 |
| CFTR | 73/55.3 | 6/7.6 | 3.163E−13 |
| ABCC6 | 48/36.4 | 2/2.5 | 1.110E−09 |
| HPP1 | 46/34.8 | 4/5.1 | 2.293E−07 |
| BCL2 | 36/27.3 | 2/2.5 | 1.457E−06 |
| ALX4 | 33/25.0 | 2/2.5 | 5.595E−06 |
| RUNX3 | 43/32.6 | 1/1.3 | 3.203E−09 |
| ITGA4 | 41/31.1 | 5/6.3 | 1.175E−05 |
| RASSF1A | 47/35.6 | 1/1.3 | 1.576E−10 |
| MYOD1 | 29/22.0 | 0/0.0 | 4.352E−07 |
| MT1A | 46/34.8 | 3/3.8 | 2.878E−08 |
| DRM | 25/18.9 | 2/2.5 | 4.354E−04 |
| BMP3B | 21/15.9 | 1/1.3 | 3.405E−04 |
| CCNA1 | 21/15.9 | 2/2.5 | 2.344E−03 |
| CDH13 | 22/16.7 | 1/1.3 | 1.940E−04 |
| RPRM | 19/14.4 | 1/1.3 | 1.098E−03 |
| MINT1 | 17/12.9 | 0/0.0 | 3.368E−04 |
| BRCA1 | 16/12.1 | 1/1.3 | 3.647E−03 |
| PTCHD2 | 7/5.3 | 1/1.3 | 2.630E−01 |
| TMS1 | 4/3.0 | 1/1.3 | 6.526E−01 |
| GSTP1 | 3/2.3 | 0/0.0 | 2.940E−01 |

TABLE 7
Methylation profiles of tested genes
in the primary and recurrent cases

| | Primary | Recurrent | |
|---|---|---|---|
| | case(s)/ | case(s)/ | |
| Gene | frequency(%) | frequency(%) | |
| Symbol | (n = 99) | (n = 33) | p value |
| SALL3 | 57/57.6 | 20/60.6 | 8.398E−01 |
| CFTR | 50/50.5 | 23/69.7 | 6.929E−02 |
| ABCC6 | 35/35.4 | 13/39.4 | 6.814E−01 |
| HPP1 | 34/34.3 | 12/36.4 | 8.358E−01 |
| BCL2 | 23/23.2 | 13/39.4 | 1.126E−01 |
| ALX4 | 23/23.2 | 10/30.3 | 4.873E−01 |
| RUNX3 | 29/29.3 | 14/42.4 | 1.992E−01 |
| ITGA4 | 31/31.3 | 10/30.3 | 1.000E+00 |
| RASSF1A | 34/34.3 | 13/39.4 | 6.759E−01 |
| MYOD1 | 22/22.2 | 7/21.2 | 1.000E+00 |
| MT1A | 34/34.3 | 12/36.4 | 8.358E−01 |
| DRM | 21/21.2 | 4/12.1 | 3.117E−01 |
| BMP3B | 17/17.2 | 4/12.1 | 5.918E−01 |
| CCNA1 | 18/18.2 | 3/9.1 | 2.791E−01 |
| CDH13 | 17/17.2 | 5/15.2 | 1.000E+00 |
| RPRM | 14/14.1 | 5/15.2 | 1.000E+00 |
| MINT1 | 11/11.1 | 6/18.2 | 3.675E−01 |
| BRCA1 | 13/13.1 | 3/9.1 | 7.599E−01 |
| PTCHD2 | 6/6.1 | 1/3.0 | 6.796E−01 |
| TMS1 | 4/4.0 | 0/0.0 | 5.716E−01 |
| GSTP1 | 2/2.0 | 1/3.0 | 1.000E+00 |

### REFERENCES

1. Jaenisch, R. and A. Bird, Epigenetic regulation of gene expression: how the genome integrates intrinsic and environmental signals. Nat Genet, 2003. 33 Suppl: p. 245-54.
2. Ting, A. H., K. M. McGarvey, and S. B. Baylin, The cancer epigenome—components and functional correlates. Genes Dev, 2006. 20(23): p. 3215-31.
3. Hanahan, D. and R. A. Weinberg, The hallmarks of cancer. Cell, 2000. 100(1): p. 57-70.
4. Bird, A., The essentials of DNA methylation. Cell, 1992. 70: p. 5-8.
5. Gaudet, F., et al., Induction of tumors in mice by genomic hypomethylation. Science, 2003. 300(5618): p. 489-92.
6. Eden, A., et al., Chromosomal instability and tumors promoted by DNA hypomethylation. Science, 2003. 300(5618): p. 455.
7. Huang, J., et al., Recurrence of DLK1 as an imprinted gene could contribute to human hepatcocellular carcinoma. Carcinogenesis, 2006. In press.
8. Belinsky, S. A., et al., Aberrant methylation of p16(INK4a) is an early event in lung cancer and a potential biomarker for early diagnosis. Proc Natl Acad Sci USA, 1998. 95(20): p. 11891-6.
9. Belinsky, S. A., Gene-promoter hypermethylation as a biomarker in lung cancer. Nat Rev Cancer, 2004. 4(9): p. 707-17.
10. Ushijima, T., T. Nakajima, and T. Maekita, DNA methylation as a marker for the past and future. J Gastroenterol, 2006. 41(5): p. 401-7.
11. Jemal, A., et al., Cancer statistics, 2006. CA Cancer J Clin, 2006. 56(2): p. 106-30.
12. Liu, J., et al., Cancer Statisitics in Shanghai, China (1972-1999). Tumor, 2004. 24(1): p. 11-13.
13. Amiel, G E. and S. P. Lerner, Combining surgery and chemotherapy for invasive bladder cancer: current and future directions. Expert Rev Anticancer Ther, 2006. 6(2): p. 281-91.
14. Eble, J., et al., Pathology and genetics of tumours of the urinary system and male genital organs. World Health Organization classification of tumours, IARC Press, Lyon (France), 2004: p. 93-109.
15. Kitamura, H. and T. Tsukamoto, Early bladder cancer: concept, diagnosis, and management. Int J Clin Oncol, 2006. 11(1): p. 28-37.
16. Kriegmair, M., et al., Detection of early bladder cancer by 5-aminolevulinic acid induced porphyrin fluorescence. J Urol, 1996. 155: p. 105-9.
17. Schneeweiss, S., M. Kriegmair, and H. Stepp, Is everything all right if nothing seems wrong? A simple method of assessing the diagnostic value of endoscopic procedures when a gold standard is absent. J Urol, 1999. 161(4): p. 1116-9.
18. Zaak, D., et al., Endoscopic detection of transitional cell carcinoma with 5-aminolevulinic acid: results of 1012 fluorescence endoscopies. Urology, 2001. 57(4): p. 690-4.
19. Wawroschek, F. and P. Rathert, [Urine cytology]. Urologe A, 1995. 34(1): p. 69-75.
20. Lin, J., et al., E-cadherin promoter polymorphism (C-160A) and risk of recurrence in patients with superficial bladder cancer. Clin Genet, 2006. 70(3): p. 240-5.
21. Schulz, W. A., Understanding urothelial carcinoma through cancer pathways. Int J Cancer, 2006. 119(7): p. 1513-8.
22. Liu, B. C. and J. R. Ehrlich, Proteomics approaches to urologic diseases. Expert Rev Proteomics, 2006. 3(3): p. 283-96.
23. Pisitkun, T., R. Johnstone, and M. A. Knepper, Discovery of Urinary Biomarkers. Mol Cell Proteomics, 2006. 5(10): p. 1760-1771.
24. Feil, G and A. Stenzl, [Tumor marker tests in bladder cancer]. Actas Urol Esp, 2006. 30(1): p. 38-45.
25. Herman, J., et al., Methylationspecific PCR: a novel PCR assay for methylation status of CpG islands. Proc Natl Acad Sci USA, 1996. 93: p. 9821-6.
26. Gonzalgo, M. L. and P. A. Jones, Rapid quantitation of methylation differences at specific sites using methylation-sensitive single nucleotide primer extension (Ms-SNuPE). Nucleic Acids Res, 1997. 25(12): p. 2529-31.
27. Kawai, J., et al., Comparison of DNA methylation patterns among mouse cell lines by restriction landmark genomic scanning. Mol Cell Biol, 1994. 14(11): p. 7421-7.
28. Huang, T. H., M. R. Perry, and D. E. Laux, Methylation profiling of CpG islands in human breast cancer cells. Hum Mol Genet, 1999. 8(3): p. 459-70.
29. Fan, J. B., et al., BeadArray-based solutions for enabling the promise of pharmacogenomics. Biotechniques, 2005. 39(4): p. 583-8.
30. Ehrich, M., et al., Quantitative high-throughput analysis of DNA methylation patterns by base-specific cleavage and mass spectrometry. Proc Natl Acad Sci USA, 2005. 102(44): p. 15785-90.
31. Yu, J., et al., Methylation profiling of twenty promoter-CpG islands of genes which may contribute to hepatocellular carcinogenesis. BMC Cancer, 2002. 2: p. 29.
32. Yu, J., et al., Methylation profiles of thirty four promoter-CpG islands and concordant methylation behaviours of sixteen genes that may contribute to carcinogenesis of astrocytoma. BMC Cancer, 2004. 4: p. 65.
33. Yu, J., et al., Methylation profiling of twenty four genes and the concordant methylation behaviours of nineteen genes that may contribute to hepatocellular carcinogenesis. Cell Res, 2003. 13(5): p. 319-33.
34. Ding, S., et al., Methylation profile of the promoter CpG islands of 14 “drug-resistance” genes in hepatocellular carcinoma. World J Gastroenterol, 2004. 10(23): p. 3433-40.
35. Li, J. L., et al., Correlation between methylation profile of promoter cpg islands of seven metastasis-associated genes and their expression states in six cell lines of liver origin. Ai Zheng, 2004. 23(9): p. 985-91.
36. Xu, X. L., et al., Methylation profile of the promoter CpG islands of 31 genes that may contribute to colorectal carcinogenesis. World J Gastroenterol, 2004. 10(23): p. 3441-54.
37. Yang, Z., et al., The methylation profiles of the promoter CpG island of nine tumor associated genes correlate with their expression in three lung cancer cell lines. Tumor, 2004. 11(3): p. 216-222.
38. Zhang, J., et al., A novel protein-DNA interaction involved with the CpG dinucleotide at −30 upstream is linked to the DNA methylation mediated transcription silencing of the MAGE-A1 gene. Cell Res, 2004. 14(4): p. 283-94.
39. Zhu, J., The altered DNA methylation pattern and its implications in liver cancer. Cell Res, 2005. 15(4): p. 272-80.
40. Zhu, J., DNA methylation and hepatocellular carcinoma. J Hepatobiliary Pancreat Surg, 2006. 13(4): p. 265-73.
41. Huang, J., et al., Recurrence of DLK1 as an imprinted gene could contribute to human hepatcocellular carcinoma. Carcinogenesis, 2007. In press.
42. Zhang, A. P., et al., The DNA methylation profile within the 5′-regulatory region of DRD2 in discordant sib pairs with schizophrenia. Schizophr Res, 2007. 90(1-3): p. 97-103.
43. Zhu, J. and X. Yao, Use of DNA methylation for cancer detection and molecular classification. J Biochem Mol Biol, 2007. 40(2): p. 135-41.
44. Hogue, M. O., et al., Quantitation of promoter methylation of multiple genes in urine DNA and bladder cancer detection. J Natl Cancer Inst, 2006. 98(14): p. 996-1004.
45. Friedrich, M. G., et al., Detection of methylated apoptosis-associated genes in urine sediments of bladder cancer patients. Clin Cancer Res, 2004. 10(22): p. 7457-65.

1. A method for diagnosing bladder cancer in a subject, comprising the following steps:
(a) collecting an urine sediment sample from said subject;
(b) determining methylation pattern of one or more genes in the sample, wherein said genes are selected from a group consisting of ABCC13, ABCC6, ABCC8, ALX4, APC, BCAR3, BCL2, BMP3B, BNIP3, BRCA1, BRCA2, CBR1, CBR3, CCNA1, CDH1, CDH13, CDKN1C, CFTR, COX2, DAPK1, DRG1, DRM, EDNRB, FADD, GALC, GSTP1, HNF3B, HPP1, HTERT, ICAM1, ITGA4, LAMA3, LITAF, MAGEA1, MDR1, MGMT, MINT1, MINT2, MT1 GMT, MINT1, MINT2, MT1A, MTSS1, MYOD1, OCLN, p14ARF, p16INK4a RASSF1A, RPRM, RUNX3, SALL3, SERPINB5, SLC29A1, STAT1, TMS1, TNFRSF10A, TNFRSF10C, TNFRSF10D, TNFRSF21, and WWOX;
(c) comparing methylation pattern of said genes in the urine sediment sample from said subject with that from normal subject, wherein the hypermethylation of one or more of genes indicates that said subject is suffering from bladder cancer.

2. The method according to claim 1, wherein said genes are selected from a group consisting of SALL3, CFTR, ABCC6, HPR1, RASSF1A, MT1A, RUNX3, ITGA4, BCL2, ALX4, MYOD1, DRM, CDH13, BMP3B, CCNA1, RPRM, MINT1, and BRCA1, and wherein the hypermethylation of at least one of said genes in the urine sediment samples indicates that said subject is suffering from bladder cancer.

3. The method according to claim 1 or 2, wherein the methylation pattern is measured by using methylation specific polymerase chain reaction or quantitative methylation specific polymerase chain reaction (QMSP).

4. The method according to any one of claims 1-3, wherein the methylation pattern of said gene is measured by using methylation-specific restriction enzyme digestion, bisulfite DNA sequencing, methylation-sensitive single nucleotide primer extension, restriction landmark genomic scanning, differential methylation hybridization, BeadArray platform technology, and a base-specific cleavage/mass spectrometry.

5. The method according to claim 1, wherein in step (b), methylation pattern of the region within the promoter CpG island of said gene are determined.

6. A kit for diagnosing bladder cancer, comprising:
(a) a reaction system for measuring methylation pattern of one or more genes in the urine sediments, wherein said genes are selected from a group consisting of ABCC13, ABCC6, ABCC8, ALX4, APC, BCAR3, BCL2, BMP3B, BNIP3, BRCA1, BRCA2, CBR1, CBR3, CCNA1, CDH1, CDH13, CDKN1C, CFTR, COX2, DAPK1, DRG1, DRM, EDNRB, FADD, GALC, GSTP1, HNF3B, HPP1, HTERT, ICAM1, ITGA4, LAMA3, LITAF, MAGEA1, MDR1, MGMT, MINT1, MINT2, MT1GMT, MINT1, MINT2, MT1A, MTS S1, MYOD1, OCLN, p14ARF, p16INK4a RASSF1A, RPRM, RUNX3, SALL3, SERPINB5, SLC29A1, STAT1, TMS1, TNFRSF10A, TNFRSF10C, TNFRSF10D, TNFRSF21, and WWOX;
(b) instructions for determining by said reaction system, and comparing the methylation pattern of one or more genes from test samples with that from normal samples, wherein hypermethylation of one or more of genes indicates that said subject is suffering from bladder cancer.

7. The kit according to claim 6, wherein said genes are selected from a group consisting of SALL3, CFTR, ABCC6, HPR1, RASSF1A, MT1A, RUNX3, ITGA4, BCL2, ALX4, MYOD1, DRM, CDH13, BMP3B, CCNA1, RPRM, MINT1, and BRCA1.

8. The kit according to claim 6, wherein said reaction system for measuring methylation pattern of the one or more genes in the urine sediment samples is selected from a group consisting of methylation-specific polymerase chain reaction system or quantitative methylation-specific polymerase chain reaction system.


SEQUENCE LISTING (236 sequences)

SEQ ID NO: 1 - DNA, 18 bp, Artificial
  primer
gcgggcggtttttattag

SEQ ID NO: 2 - DNA, 18 bp, Artificial
  Primer
caaaaactcgtccgtcca

SEQ ID NO: 3 - DNA, 17 bp, Artificial
  Primer
tgggtttgtggggtgtt

SEQ ID NO: 4 - DNA, 22 bp, Artificial
  Primer
acaaaaactcatccatccacat

SEQ ID NO: 5 - DNA, 15 bp, Artificial
  Primer
ggcgttcggggagtt

SEQ ID NO: 6 - DNA, 18 bp, Artificial
  Primer
cgacctcgacccgataat

SEQ ID NO: 7 - DNA, 18 bp, Artificial
  Primer
aggtgtttggggagttgg

SEQ ID NO: 8 - DNA, 22 bp, Artificial
  Primer
tctcaacctcaacccaataatc

SEQ ID NO: 9 - DNA, 19 bp, Artificial
  Primer
gacgtgcggtattacgttg

SEQ ID NO: 10 - DNA, 18 bp, Artificial
  Primer
acaaaaacgcgacaaacg

SEQ ID NO: 11 - DNA, 18 bp, Artificial
  Primer
aggatggggaaggtgatg

SEQ ID NO: 12 - DNA, 24 bp, Artificial
  Primer
aaaacaaaaacacaacaaacacac

SEQ ID NO: 13 - DNA, 20 bp, Artificial
  Primer
gagtttgaggttgtcgttcg

SEQ ID NO: 14 - DNA, 20 bp, Artificial
  Primer
aacccgttacgacgctaaac

SEQ ID NO: 15 - DNA, 18 bp, Artificial
  Primer
ttgtttgggggtgttttg

SEQ ID NO: 16 - DNA, 22 bp, Artificial
  Primer
aaaccaaacccattacaacact

SEQ ID NO: 17 - DNA, 18 bp, Artificial
  Primer
tattgcggagtgcgggtc

SEQ ID NO: 18 - DNA, 18 bp, Artificial
  Primer
tcgacgaactcccgacga

SEQ ID NO: 19 - DNA, 24 bp, Artificial
  Primer
gtgttttattgtggagtgtgggtt

SEQ ID NO: 20 - DNA, 22 bp, Artificial
  Primer
ccaatcaacaaactcccaacaa

SEQ ID NO: 21 - DNA, 18 bp, Artificial
  Primer
gcgtttcgggaggaatag

SEQ ID NO: 22 - DNA, 19 bp, Artificial
  Primer
actacgaaacgcaccgact

SEQ ID NO: 23 - DNA, 18 bp, Artificial
  Primer
tgggtgtgtggtggagat

SEQ ID NO: 24 - DNA, 25 bp, Artificial
  Primer
ctacaaaacacaccaactaaacaca

SEQ ID NO: 25 - DNA, 18 bp, Artificial
  Primer
gaagtcgtcgtcggtttg

SEQ ID NO: 26 - DNA, 15 bp, Artificial
  Primer
cccgcaccgaacatc

SEQ ID NO: 27 - DNA, 19 bp, Artificial
  Primer
ttgttgttggtttggtgga

SEQ ID NO: 28 - DNA, 20 bp, Artificial
  Primer
cccacaccaaacatcttctc

SEQ ID NO: 29 - DNA, 18 bp, Artificial
  Primer
gcggtaaagggtcgaagt

SEQ ID NO: 30 - DNA, 17 bp, Artificial
  Primer
aactcgaaccgccgata

SEQ ID NO: 31 - DNA, 19 bp, Artificial
  Primer
tgagggtggtaaagggttg

SEQ ID NO: 32 - DNA, 22 bp, Artificial
  Primer
aaaaactcaaaccaccaatacc

SEQ ID NO: 33 - DNA, 18 bp, Artificial
  Primer
tcgttcggtttcgttttg

SEQ ID NO: 34 - DNA, 18 bp, Artificial
  Primer
acgctccgttctacgaca

SEQ ID NO: 35 - DNA, 25 bp, Artificial
  Primer
gttgtagatttgtttggttttgttt

SEQ ID NO: 36 - DNA, 21 bp, Artificial
  Primer
acatcccaaacactccattct

SEQ ID NO: 37 - DNA, 25 bp, Artificial
  Primer
ggttaatttagagtttcgagagacg

SEQ ID NO: 38 - DNA, 25 bp, Artificial
  Primer
tcaacgaactcacgccgcgcaatcg

SEQ ID NO: 39 - DNA, 25 bp, Artificial
  Primer
ggttaatttagagttttgagagatg

SEQ ID NO: 40 - DNA, 25 bp, Artificial
  Primer
tcaacaaactcacaccacacaatca

SEQ ID NO: 41 - DNA, 18 bp, Artificial
  Primer
gcggagattgcgttattg

SEQ ID NO: 42 - DNA, 18 bp, Artificial
  Primer
ccgaacccgtttccttac

SEQ ID NO: 43 - DNA, 18 bp, Artificial
  Primer
tggaggtggaagttgtgg

SEQ ID NO: 44 - DNA, 21 bp, Artificial
  Primer
ctccaaacccatttccttact

SEQ ID NO: 45 - DNA, 17 bp, Artificial
  Primer
tcgtatttcggcgaggt

SEQ ID NO: 46 - DNA, 18 bp, Artificial
  Primer
aaaccccgcaacgtattc

SEQ ID NO: 47 - DNA, 16 bp, Artificial
  Primer
ttggtggggaggggta

SEQ ID NO: 48 - DNA, 18 bp, Artificial
  Primer
aaaccccacaacatattc

SEQ ID NO: 49 - DNA, 22 bp, Artificial
  Primer
cgtagattatttcgcggtttag

SEQ ID NO: 50 - DNA, 19 bp, Artificial
  Primer
gaaccgaacttcgaaccac

SEQ ID NO: 51 - DNA, 19 bp, Artificial
  Primer
gggtgtagtgtgggtaggg

SEQ ID NO: 52 - DNA, 21 bp, Artificial
  Primer
aaaccaaacttcaaaccacct

SEQ ID NO: 53 - DNA, 18 bp, Artificial
  Primer
tcgtcgcgttttagtcgt

SEQ ID NO: 54 - DNA, 18 bp, Artificial
  Primer
acccgttctcccaacaac

SEQ ID NO: 55 - DNA, 25 bp, Artificial
  Primer
gggtagttttgttgtgttttagttg

SEQ ID NO: 56 - DNA, 20 bp, Artificial
  Primer
aaccactaacaaccccctct

SEQ ID NO: 57 - DNA, 20 bp, Artificial
  Primer
gtgggcgggtcgttagtttc

SEQ ID NO: 58 - DNA, 26 bp, Artificial
  Primer
ctcacaaatactttacaattccgacg

SEQ ID NO: 59 - DNA, 23 bp, Artificial
  Primer
ggtgggtgggttgttagttttgt

SEQ ID NO: 60 - DNA, 26 bp, Artificial
  Primer
aactcacaaatctttacaattccaac

SEQ ID NO: 61 - DNA, 20 bp, Artificial
  Primer
tcgcggggttcgtttttcgc

SEQ ID NO: 62 - DNA, 22 bp, Artificial
  Primer
gacgttttcattcatacacgcg

SEQ ID NO: 63 - DNA, 20 bp, Artificial
  Primer
ttgtggggtttgttttttgt

SEQ ID NO: 64 - DNA, 21 bp, Artificial
  Primer
aacttttcattcatacacaca

SEQ ID NO: 65 - DNA, 18 bp, Artificial
  Primer
ggttcggttttcgcgtat

SEQ ID NO: 66 - DNA, 18 bp, Artificial
  Primer
aaaacgaacgtcgcgata

SEQ ID NO: 67 - DNA, 21 bp, Artificial
  Primer
tttgttgtggtttggtttttg

SEQ ID NO: 68 - DNA, 26 bp, Artificial
  Primer
aacaaacatcacaatatcacattacc

SEQ ID NO: 69 - DNA, 19 bp, Artificial
  Primer
agaggtcgcgattgtcgtt

SEQ ID NO: 70 - DNA, 21 bp, Artificial
  Primer
cgactttctccacccactacg

SEQ ID NO: 71 - DNA, 25 bp, Artificial
  Primer
ttaaagagaggttgtgattgttgtt

SEQ ID NO: 72 - DNA, 18 bp, Artificial
  Primer
tccttcactccctcacca

SEQ ID NO: 73 - DNA, 18 bp, Artificial
  Primer
gttcgtcgttgcgatgtt

SEQ ID NO: 74 - DNA, 20 bp, Artificial
  Primer
ccaaactctttcccaaatca

SEQ ID NO: 75 - DNA, 22 bp, Artificial
  Primer
ttgtttgttgttgtgatgtttg

SEQ ID NO: 76 - DNA, 20 bp, Artificial
  Primer
tccaaactctttcccaaatc

SEQ ID NO: 77 - DNA, 20 bp, Artificial
  Primer
tcggtaattcgtagcggtag

SEQ ID NO: 78 - DNA, 18 bp, Artificial
  Primer
tactcacccgaacgccta

SEQ ID NO: 79 - DNA, 22 bp, Artificial
  Primer
gggatttggtaatttgtagtgg

SEQ ID NO: 80 - DNA, 23 bp, Artificial
  Primer
cctaactactcacccaaacacct

SEQ ID NO: 81 - DNA, 18 bp, Artificial
  Primer
ggtgcggagtatgagtcg

SEQ ID NO: 82 - DNA, 18 bp, Artificial
  Primer
ccgcgaaccaatacgata

SEQ ID NO: 83 - DNA, 19 bp, Artificial
  Primer
gtgaggaataggggtgtgg

SEQ ID NO: 84 - DNA, 23 bp, Artificial
  Primer
cccacaaaccaatacaatatcat

SEQ ID NO: 85 - DNA, 18 bp, Artificial
  Primer
tcggtttcgttgatttcg

SEQ ID NO: 86 - DNA, 19 bp, Artificial
  Primer
aaactaccgcgcgtaaaac

SEQ ID NO: 87 - DNA, 20 bp, Artificial
  Primer
ttgagttttggtggttttgg

SEQ ID NO: 88 - DNA, 19 bp, Artificial
  Primer
aaactaccacacataaaac

SEQ ID NO: 89 - DNA, 18 bp, Artificial
  Primer
tagggcgcgttcgtatag

SEQ ID NO: 90 - DNA, 18 bp, Artificial
  Primer
ccactaacgcgcaaactt

SEQ ID NO: 91 - DNA, 24 bp, Artificial
  Primer
tgtgtttgtatagatttggaggtg

SEQ ID NO: 92 - DNA, 24 bp, Artificial
  Primer
ttcccactaacacacaaacttaaa

SEQ ID NO: 93 - DNA, 16 bp, Artificial
  Primer
cgtgacgttcgggttg

SEQ ID NO: 94 - DNA, 17 bp, Artificial
  Primer
cctacgcccgacgtatc

SEQ ID NO: 95 - DNA, 22 bp, Artificial
  Primer
tggatttggtagaggtgtgatt

SEQ ID NO: 96 - DNA, 24 bp, Artificial
  Primer
tacacctacacccaacatatcatc

SEQ ID NO: 97 - DNA, 19 bp, Artificial
  Primer
ggtgacgtcggaagagaag

SEQ ID NO: 98 - DNA, 18 bp, Artificial
  Primer
ccgccacgataaatacga

SEQ ID NO: 99 - DNA, 25 bp, Artificial
  Primer
ttattaggtgatgttggaagagaag

SEQ ID NO: 100 - DNA, 20 bp, Artificial
  Primer
aaaaacaaatcccatcacca

SEQ ID NO: 101 - DNA, 18 bp, Artificial
  Primer
gcgatttcggggatttta

SEQ ID NO: 102 - DNA, 18 bp, Artificial
  Primer
acgacgacgaaactccaa

SEQ ID NO: 103 - DNA, 18 bp, Artificial
  Primer
gttggggatttgggaaag

SEQ ID NO: 104 - DNA, 23 bp, Artificial
  Primer
tataaaaataatcccaccccact

SEQ ID NO: 105 - DNA, 19 bp, Artificial
  Primer
cgttcgttgttgtttttgc

SEQ ID NO: 106 - DNA, 19 bp, Artificial
  Primer
aaccgtcgaccgctactaa

SEQ ID NO: 107 - DNA, 18 bp, Artificial
  Primer
gggagaagtgtggggtgt

SEQ ID NO: 108 - DNA, 22 bp, Artificial
  Primer
cccaaccatcaaccactactaa

SEQ ID NO: 109 - DNA, 18 bp, Artificial
  Primer
aagaggggcgttagttcg

SEQ ID NO: 110 - DNA, 16 bp, Artificial
  Primer
cgctcgcaaacgctaa

SEQ ID NO: 111 - DNA, 19 bp, Artificial
  Primer
atgtgtggaagaggggtgt

SEQ ID NO: 112 - DNA, 22 bp, Artificial
  Primer
cactcacaaacactaacccaaa

SEQ ID NO: 113 - DNA, 15 bp, Artificial
  Primer
gcgtcgcgaggagag

SEQ ID NO: 114 - DNA, 18 bp, Artificial
  Primer
aattcgcgaacacaaacg

SEQ ID NO: 115 - DNA, 18 bp, Artificial
  Primer
ggggttgtggaaaggaag

SEQ ID NO: 116 - DNA, 21 bp, Artificial
  Primer
aaccacacttcccacataaca

SEQ ID NO: 117 - DNA, 18 bp, Artificial
  Primer
tagcgcggtgtagatcgt

SEQ ID NO: 118 - DNA, 22 bp, Artificial
  Primer
cgaactaacaaaatacccgaac

SEQ ID NO: 119 - DNA, 17 bp, Artificial
  Primer
ttgggaaatgggaggtg

SEQ ID NO: 120 - DNA, 24 bp, Artificial
  Primer
tccaaactaacaaaatacccaaac

SEQ ID NO: 121 - DNA, 18 bp, Artificial
  Primer
gacgcgagttttgcgtag

SEQ ID NO: 122 - DNA, 18 bp, Artificial
  Primer
taaaataccgcgcactcg

SEQ ID NO: 123 - DNA, 19 bp, Artificial
  Primer
gggaggtttgggttaggat

SEQ ID NO: 124 - DNA, 24 bp, Artificial
  Primer
caacctaaaataccacacactcac

SEQ ID NO: 125 - DNA, 18 bp, Artificial
  Primer
ttcgttcgcgaagtttgt

SEQ ID NO: 126 - DNA, 17 bp, Artificial
  Primer
taaacgacgccgaaacc

SEQ ID NO: 127 - DNA, 20 bp, Artificial
  Primer
tgtgttttgtgtgggagaga

SEQ ID NO: 128 - DNA, 21 bp, Artificial
  Primer
aaacaacaccaaaaccactcc

SEQ ID NO: 129 - DNA, 18 bp, Artificial
  Primer
cggtcgggtttttacgtt

SEQ ID NO: 130 - DNA, 17 bp, Artificial
  Primer
acctcccgactcgacaa

SEQ ID NO: 131 - DNA, 20 bp, Artificial
  Primer
gggaggttggattttgtttt

SEQ ID NO: 132 - DNA, 20 bp, Artificial
  Primer
caaacctcccaactcaacaa

SEQ ID NO: 133 - DNA, 20 bp, Artificial
  Primer
gttcggtcgaaggaatttga

SEQ ID NO: 134 - DNA, 20 bp, Artificial
  Primer
ccacaaccctccctcttaaa

SEQ ID NO: 135 - DNA, 20 bp, Artificial
  Primer
gtttggttgaaggaatttga

SEQ ID NO: 136 - DNA, 20 bp, Artificial
  Primer
acccacaaccctccctctta

SEQ ID NO: 137 - DNA, 18 bp, Artificial
  Primer
ttgggggtttggtagcgc

SEQ ID NO: 138 - DNA, 21 bp, Artificial
  Primer
ctctctaaacccgcgaacgat

SEQ ID NO: 139 - DNA, 19 bp, Artificial
  Primer
gttgggggtttggtagtgt

SEQ ID NO: 140 - DNA, 22 bp, Artificial
  Primer
actctctaaacccacaaacaat

SEQ ID NO: 141 - DNA, 18 bp, Artificial
  Primer
agcgtcgttgttttgtgc

SEQ ID NO: 142 - DNA, 18 bp, Artificial
  Primer
cgctttcaaaaccactcg

SEQ ID NO: 143 - DNA, 23 bp, Artificial
  Primer
ttggtagtgttgttgttttgtgt

SEQ ID NO: 144 - DNA, 18 bp, Artificial
  Primer
catcctacaacccccaca

SEQ ID NO: 145 - DNA, 20 bp, Artificial
  Primer
tgttggtggattttggattt

SEQ ID NO: 146 - DNA, 24 bp, Artificial
  Primer
aacaacaattccatacacctttct

SEQ ID NO: 147 - DNA, 18 bp, Artificial
  Primer
agttcgttggcggatttt

SEQ ID NO: 148 - DNA, 21 bp, Artificial
  Primer
cccgaaataataacgacgatt

SEQ ID NO: 149 - DNA, 18 bp, Artificial
  Primer
ttcgaagcgtttgtttgg

SEQ ID NO: 150 - DNA, 18 bp, Artificial
  Primer
cgcctaacctaacgcaca

SEQ ID NO: 151 - DNA, 25 bp, Artificial
  Primer
tatttttgaagtgtttgtttggtgt

SEQ ID NO: 152 - DNA, 21 bp, Artificial
  Primer
tccctctcccctctaaacttc

SEQ ID NO: 153 - DNA, 20 bp, Artificial
  Primer
taaggttgggttttcggaac

SEQ ID NO: 154 - DNA, 20 bp, Artificial
  Primer
aaatacgaaccacgaaacca

SEQ ID NO: 155 - DNA, 20 bp, Artificial
  Primer
taaggttgggtttttggaat

SEQ ID NO: 156 - DNA, 21 bp, Artificial
  Primer
ctcccctaaatacaaaccaca

SEQ ID NO: 157 - DNA, 19 bp, Artificial
  Primer
tgatatttcggtcgggagt

SEQ ID NO: 158 - DNA, 18 bp, Artificial
  Primer
aaatacaacgcgctcgaa

SEQ ID NO: 159 - DNA, 21 bp, Artificial
  Primer
ggtgatattttggttgggagt

SEQ ID NO: 160 - DNA, 25 bp, Artificial
  Primer
aaatacaacacactcaaaaacctct

SEQ ID NO: 161 - DNA, 18 bp, Artificial
  Primer
gacggttttcgacggttt

SEQ ID NO: 162 - DNA, 18 bp, Artificial
  Primer
gcccgaaaccgaatacac

SEQ ID NO: 163 - DNA, 22 bp, Artificial
  Primer
atttgatggtttttgatggttt

SEQ ID NO: 164 - DNA, 22 bp, Artificial
  Primer
cacacacatactcatcctcaca

SEQ ID NO: 165 - DNA, 18 bp, Artificial
  Primer
tgcgttcgttaggtgagc

SEQ ID NO: 166 - DNA, 20 bp, Artificial
  Primer
cgaatcccaactcgaaaacg

SEQ ID NO: 167 - DNA, 24 bp, Artificial
  Primer
gttaggtgtgtttgttaggtgagt

SEQ ID NO: 168 - DNA, 21 bp, Artificial
  Primer
cacacctctctaattcccaca

SEQ ID NO: 169 - DNA, 20 bp, Artificial
  Primer
gtcgagttcggttttggagg

SEQ ID NO: 170 - DNA, 19 bp, Artificial
  Primer
aaaaccacaacgacgaacg

SEQ ID NO: 171 - DNA, 21 bp, Artificial
  Primer
tgagtttggttttggaggtgg

SEQ ID NO: 172 - DNA, 22 bp, Artificial
  Primer
aaccacaacaacaaacacccct

SEQ ID NO: 173 - DNA, 24 bp, Artificial
  Primer
ttattagagggtggggcggatcgc

SEQ ID NO: 174 - DNA, 20 bp, Artificial
  Primer
accccgaaccgcgaccgtaa

SEQ ID NO: 175 - DNA, 24 bp, Artificial
  Primer
ttattagagggtggggtggattgt

SEQ ID NO: 176 - DNA, 22 bp, Artificial
  Primer
caaccccaaaccacaaccataa

SEQ ID NO: 177 - DNA, 18 bp, Artificial
  Primer
tttcgcggtcgttttaga

SEQ ID NO: 178 - DNA, 18 bp, Artificial
  Primer
ccgcccacgtacgtataa

SEQ ID NO: 179 - DNA, 23 bp, Artificial
  Primer
tggatagtgttttgtggttgttt

SEQ ID NO: 180 - DNA, 23 bp, Artificial
  Primer
ccacccacatacatataaaccat

SEQ ID NO: 181 - DNA, 20 bp, Artificial
  Primer
gtgttaacgcgttgcgtatc

SEQ ID NO: 182 - DNA, 21 bp, Artificial
  Primer
aaccccgcgaactaaaaacga

SEQ ID NO: 183 - DNA, 23 bp, Artificial
  Primer
tttggttggagtgtgttaatgtg

SEQ ID NO: 184 - DNA, 23 bp, Artificial
  Primer
caaaccccacaaactaaaaacaa

SEQ ID NO: 185 - DNA, 23 bp, Artificial
  Primer
tgagcgtttattcgtagattagc

SEQ ID NO: 186 - DNA, 17 bp, Artificial
  Primer
gaacgaacgccgaaaac

SEQ ID NO: 187 - DNA, 18 bp, Artificial
  Primer
gtggtggtgttggaggaa

SEQ ID NO: 188 - DNA, 23 bp, Artificial
  Primer
tcaaacaaacaccaaaaacaaac

SEQ ID NO: 189 - DNA, 20 bp, Artificial
  Primer
gaggggcggtcgtacgcggg

SEQ ID NO: 190 - DNA, 24 bp, Artificial
  Primer
aaaacgaccgacgcgaacgcctcc

SEQ ID NO: 191 - DNA, 20 bp, Artificial
  Primer
gaggggtggttgtatgtggg

SEQ ID NO: 192 - DNA, 24 bp, Artificial
  Primer
aaaacaaccaacacaaacacctcc

SEQ ID NO: 193 - DNA, 18 bp, Artificial
  Primer
gttcgcgtagtcgtcgtc

SEQ ID NO: 194 - DNA, 18 bp, Artificial
  Primer
tactcgaaaaccccgtca

SEQ ID NO: 195 - DNA, 24 bp, Artificial
  Primer
gtggtttgtgtagttgttgttgtt

SEQ ID NO: 196 - DNA, 19 bp, Artificial
  Primer
cccaaccctcaccatactc

SEQ ID NO: 197 - DNA, 16 bp, Artificial
  Primer
tttgcgtgggtcgaga

SEQ ID NO: 198 - DNA, 16 bp, Artificial
  Primer
gcctcgacgacactcc

SEQ ID NO: 199 - DNA, 19 bp, Artificial
  Primer
ttttgtgtgggttgagagg

SEQ ID NO: 200 - DNA, 15 bp, Artificial
  Primer
caccccaccccacct

SEQ ID NO: 201 - DNA, 18 bp, Artificial
  Primer
aaggcgtcggtcgttagt

SEQ ID NO: 202 - DNA, 18 bp, Artificial
  Primer
tataaaccgccgaacgaa

SEQ ID NO: 203 - DNA, 20 bp, Artificial
  Primer
tgggtgtttaaaggtgttgg

SEQ ID NO: 204 - DNA, 23 bp, Artificial
  Primer
accaatataaaccaccaaacaaa

SEQ ID NO: 205 - DNA, 18 bp, Artificial
  Primer
gtcgttcggtgattggtg

SEQ ID NO: 206 - DNA, 18 bp, Artificial
  Primer
aacgaaaacgcgacgata

SEQ ID NO: 207 - DNA, 22 bp, Artificial
  Primer
tgtttaattggttgagtgtgga

SEQ ID NO: 208 - DNA, 27 bp, Artificial
  Primer
aaactaaacaaaaacacaacaatacaa

SEQ ID NO: 209 - DNA, 20 bp, Artificial
  Primer
gcgttttatttcgtttcgtc

SEQ ID NO: 210 - DNA, 18 bp, Artificial
  Primer
cacgataaacccgaacca

SEQ ID NO: 211 - DNA, 21 bp, Artificial
  Primer
gttgggtatttggagggtagt

SEQ ID NO: 212 - DNA, 21 bp, Artificial
  Primer
cacaataaacccaaaccaaaa

SEQ ID NO: 213 - DNA, 19 bp, Artificial
  Primer
ttgtagcggggtgagcggc

SEQ ID NO: 214 - DNA, 22 bp, Artificial
  Primer
aacgtccataaacaacaacgcg

SEQ ID NO: 215 - DNA, 21 bp, Artificial
  Primer
ggttgtagtggggtgagtggt

SEQ ID NO: 216 - DNA, 25 bp, Artificial
  Primer
caaaacatccataaacaacaacaca

SEQ ID NO: 217 - DNA, 18 bp, Artificial
  Primer
gtttttcggtcgggagtt

SEQ ID NO: 218 - DNA, 19 bp, Artificial
  Primer
actcgcccgataataacga

SEQ ID NO: 219 - DNA, 18 bp, Artificial
  Primer
tgtttggtggatggatgg

SEQ ID NO: 220 - DNA, 27 bp, Artificial
  Primer
actaaatcactcacccaataataacaa

SEQ ID NO: 221 - DNA, 17 bp, Artificial
  Primer
agcgtttcggtcgtttg

SEQ ID NO: 222 - DNA, 18 bp, Artificial
  Primer
taccgtatccccgtctcc

SEQ ID NO: 223 - DNA, 20 bp, Artificial
  Primer
tggttgaggtagggtgtgat

SEQ ID NO: 224 - DNA, 21 bp, Artificial
  Primer
taccatatccccatctcccta

SEQ ID NO: 225 - DNA, 18 bp, Artificial
  Primer
gaatcgcgacgatgaaga

SEQ ID NO: 226 - DNA, 17 bp, Artificial
  Primer
cacgcgcacaaactacg

SEQ ID NO: 227 - DNA, 24 bp, Artificial
  Primer
agaattgtgatgatgaagatgatg

SEQ ID NO: 228 - DNA, 25 bp, Artificial
  Primer
aacctttacacacacacaaactaca

SEQ ID NO: 229 - DNA, 23 bp, Artificial
  Primer
ttgtttagcgtcgtatttatcgt

SEQ ID NO: 230 - DNA, 18 bp, Artificial
  Primer
tcctcaaccgctatcgaa

SEQ ID NO: 231 - DNA, 21 bp, Artificial
  Primer
tttttgggttgggagtttatt

SEQ ID NO: 232 - DNA, 25 bp, Artificial
  Primer
taattctcctcaaccactatcaaaa

SEQ ID NO: 233 - DNA, 18 bp, Artificial
  Primer
gcgatattgcggagattg

SEQ ID NO: 234 - DNA, 16 bp, Artificial
  Primer
ccctatcgcccgctac

SEQ ID NO: 235 - DNA, 24 bp, Artificial
  Primer
ttgtggagattggattttagtttt

SEQ ID NO: 236 - DNA, 21 bp, Artificial
  Primer
ccctatcacccactaccaaat